SEQUENCE LISTING (6 sequences)

SEQ ID NO: 1 - RNA, 19 nt, Artificial Sequence
  Description of Artificial Sequence Synthetic oligonucleotide
ggagaaagccgucuuaauu

SEQ ID NO: 2 - RNA, 19 nt, Artificial Sequence
  Description of Artificial Sequence Synthetic oligonucleotide
ggcaaagacuauaauguaa

SEQ ID NO: 3 - RNA, 19 nt, Artificial Sequence
  Description of Artificial Sequence Synthetic oligonucleotide
uaagggucuuuacggaaua

SEQ ID NO: 4 - RNA, 19 nt, Artificial Sequence
  Description of Artificial Sequence Synthetic oligonucleotide
aaagucuucugaugucaua

SEQ ID NO: 5 - protein, 332 aa, Homo sapiens
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 6 - protein, 274 aa, Homo sapiens
MATLKDQLIYNLLKEEQTPQNKITVVGVGAVGMACAISILMKDLADELALVDVIEDKLKGEMMDLQHGSLFLRTPKIVSGKVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

(19) United States
(12) Patent Application Publication
(10) Pub. No.: US 2012/0003156 A1
(43) Pub. Date: Jan. 5, 2012

(54) METHODS FOR TREATING NEOPLASIA BY INHIBITING LACTATE DEHYDROGENASE AND/OR NICOTINAMIDE PHOSPHORIBOSYLTRANSFERASE

(75) Inventors: Chi V. Dang, Baltimore, MD (US); Quy H. Le, Baltimore, MD (US); Ramani Dinavahi, Millersville, MD (US)

(73) Assignee: The Johns Hopkins University, Baltimore, MD (US)

(21) Appl. No.: 13/002,202

(22) PCT Filed: Jul. 1, 2009
(86) PCT No.: PCT/US2009/003930

§ 371 Date: Sep. 14, 2011

Related U.S. Application Data
(60) Provisional application No. 61/133,673, filed on Jul. 1, 2008.
(60) Provisional application No. 61/142,985, filed on Jan. 7, 2009.
(60) Provisional application No. 61/143,257, filed on Jan. 8, 2009.

(51) Int. Cl. A61K 49/00; C07C 65/11; A61P 35/00; A61K 31/4545; C12Q 1/32; A61K 31/713; A61K 31/192

(52) U.S. Cl. 424/9.1; 514/44 A; 562/467; 514/569; 514/318; 435/26

(57) ABSTRACT

The invention provides compositions for the diagnosis or treatment of neoplasias, including lymphomas, leukemias, brain cancers (e. glioblastomas, medulloblastomas), breast cancer, colon cancer, and pancreatic cancer, and methods of use therefor.

### CROSS-REFERENCE TO RELATED APPLICATION

This application claims the benefit of the following U.S. Provisional Application No. 61/133,673, which was filed Jul. 1, 2008, 61/142,985, which was filed Jan. 7, 2009, 61/143,257, which was filed Jan. 8, 2009, and the provisional application entitled “Treatment for a variety of cancer types through combinatorial inhibition of lactate dehydrogenase and nicotinamide phosphoribosyltransferase,” filed Jun. 5, 2009, the entire contents of which are incorporated herein by reference.

### STATEMENT OF RIGHTS TO INVENTIONS MADE UNDER FEDERALLY SPONSORED RESEARCH

This work was supported by the following grants from the National Institutes of Health, Grant Nos: The government has certain rights in the invention.

### BACKGROUND OF THE INVENTION

In normal cells and tissues, generation of ATP through oxidative phosphorylation in the mitochondria produces more ATP molecules from a given amount of glucose than glycolysis. When a cells ability to generate ATP through mitochondrial oxidative phosphorylation is compromised, cells adapt by increasing their glycolytic activity. The metabolism of neoplasias differs from that observed in normal cells and tissues. A variety of neoplasias display increased glycolytic activity. This characteristic of many neoplasias has been exploited for diagnostic and prognostic purposes. For example, metabolic imaging with fluorine-18 fluorodeoxyglucose positron emission tomography can be used for the diagnosis, staging, and monitoring of a variety of cancers, including non-Hodgkins lymphoma (NHL). NHL is a cancer that initially effects lymphoid tissues, although it can spread to other organs. Patients with advanced disease usually receive treatment with the drugs doxorubicin, bleomycin, vinblastine, and dacarbazine (ABVD). However, a subset of patients fail to respond to this treatment regimen. In these patients, the disease continues to progress despite therapy. Such patients can be identified using a PET scan. Typically imaging is performed after two rounds of chemotherapy. Patients whose NHL's are PET-positive after two rounds of chemotherapy are less likely to survive, and more likely to have relapses. Conventional methods for treating such patients are inadequate. Thus, improved therapeutic compositions and methods are urgently required for the treatment of lymphomas and other neoplasias characterized by a glycolytic metabolism.

### SUMMARY OF THE INVENTION

As described below, the present invention features compositions and methods for the diagnosis, treatment or prevention of neoplasias characterized by a glycolytic metabolism.

In one aspect, the invention provides a composition for the treatment of neoplasia, the composition containing an effective amount of a compound of Formula III or IV,

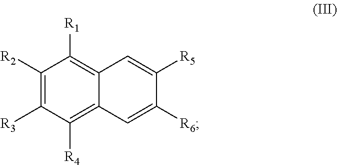

where,

R₁ is an optionally substituted alkyl or an optionally substituted aralkyl;

R₂ is H, —C(O)R′, —OR″, or —NR′R″;

R₃ is H, —C(O)R′, —OR″, or —NR′R″;

R₄ is —C(O)R′ or —OR″;

R₅ is an optionally substituted alkyl, an optionally substituted aryl, an optionally substituted heteroaryl, an optionally substituted aralkyl, or an optionally substituted heteroaralkyl;

R₆ is an optionally substituted aralkyl or an optionally substituted heteroaralkyl;

R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R″ for each occurrence, is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl; and

R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl.

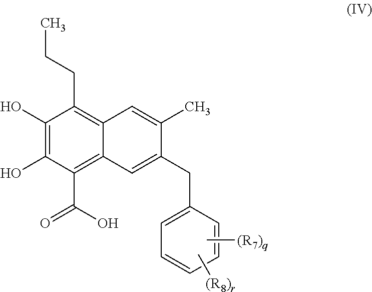

where,
1. each R₇ and R₈ is independently:
2. (i) an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
3. (ii) an optionally substituted haloalkyl, cyano, nitro, azido, or halo;
4. (iii) OR′, SR′, S(O)R′, S(O)₂R′, N(R′)₂, C(O)R′, C(S)R′, C(S)NR′R′, C(NR′)R′, C(NR′)NR′R′, C(O)NR′R′, C(O)NR′OR′, C(O)OR′, OC(O)R′, OC(O)OR′, NR′C(O)NR′R′, NR′C(S)NR′R′, NR′C(O)R′, NR′C(O)OR′, OC(O)NR′R′, or S(O)ᵣNR′R′; or
5. (iv) R₇ and R₈ may together with the carbon atoms to which each is attached, form a fussed bicyclic aryl, heteroaryl, cycloalkyl, or heterocycloalkyl, each of which may be optionally substituted;

R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

q is 0, 1, 2, or 3; and

r is 0, 1, or 2.

In another aspect, the invention provides a method for treating neoplasia in a subject, the method involving administering to the subject an effective amount of a compound that is

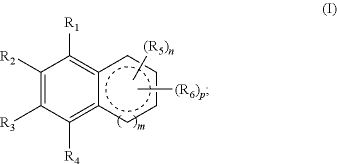

where,

R₁ is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, an optionally substituted heteroaralkyl, —C(O)R′, —OR″, —S(O)ₘR′, —NR′R″, or haloalkyl;

R₂ is H, —C(O)R′, —OR″, —S(O)ₘR′, —NR′R″, nitro, cyano, halogen, or haloalkyl;

R₃ is H, —C(O)R′, —OR″, —S(O)ₘR′, —NR′R″, nitro, cyano, halogen, or haloalkyl;

R₄ is H, an optionally substituted alkyl, an optionally substituted aryl, an optionally substituted heteroaryl, an optionally substituted aralkyl, an optionally substituted heteroaralkyl, —C(O)R′, —OR″, —S(O)ₘR′, or —NR′R″;

each R₅ is independently an optionally substituted alkyl, an optionally substituted aryl, an optionally substituted heteroaryl, an optionally substituted aralkyl, or an optionally substituted heteroaralkyl;

each R₆ is independently H, an optionally substituted alkyl, an optionally substituted aryl, an optionally substituted heteroaryl, an optionally substituted aralkyl, or an optionally substituted heteroaralkyl;

R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R″ for each occurrence, is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

m is 0, 1, or 2;

n is 1 or 2; and

p is 1 or 2.

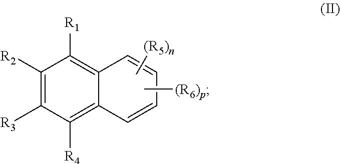

where,

R₁ is an optionally substituted alkyl, an optionally substituted aryl, an optionally substituted heteroaryl, an optionally substituted aralkyl, or an optionally substituted heteroaralkyl;

R₂ is H, —C(O)R′, —OR″, —NR′R″, halogen, or haloalkyl;

R₃ is H, —C(O)R′, —OR″, —NR′R″, halogen, or haloalkyl;

R₄ is —C(O)R′, —OR″, —S(O)ₘR′, or —NR′R″;

each R₅ is independently an optionally substituted alkyl, an optionally substituted aryl, an optionally substituted heteroaryl, an optionally substituted aralkyl, or an optionally substituted heteroaralkyl;

each R₆ is independently H, an optionally substituted alkyl, an optionally substituted aryl, an optionally substituted heteroaryl, an optionally substituted aralkyl, or an optionally substituted heteroaralkyl;

R′ for each occurrence, is H, —C(O)R′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R″ for each occurrence, is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

n is 1 or 2; and

p is 1 or 2.

In yet another aspect, the invention provides a method for treating neoplasia in a subject, the method involving administering to the subject an effective amount of a compound that is

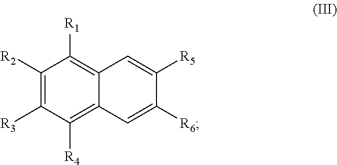

where,

R₁ is an optionally substituted alkyl or an optionally substituted aralkyl;

R₂ is H, —C(O)R′, —OR″, or —NR′R″;

R₃ is H, —C(O)R′, —OR″, or —NR′R″;

R₄ is —C(O)R′ or —OR″;

R₅ is an optionally substituted alkyl, an optionally substituted aryl, an optionally substituted heteroaryl, an optionally substituted aralkyl, or an optionally substituted heteroaralkyl;

R₆ is an optionally substituted alkyl, an optionally substituted aryl, an optionally substituted heteroaryl, an optionally substituted aralkyl, or an optionally substituted heteroaralkyl;

R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R″ for each occurrence, is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl; and

R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl.

In yet another aspect, the invention provides a method for treating neoplasia in a subject, the method involving administering to the subject an effective amount of a compound that is

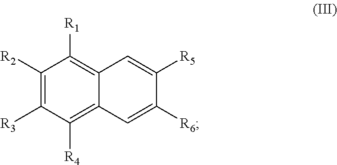

where,

R₁ is an optionally substituted alkyl or an optionally substituted aralkyl;

R₂ is H, —C(O)R′, —OR″, or —NR′R″;

R₃ is H, —C(O)R′, —OR″, or —NR′R″;

R₄ is —C(O)R′ or —OR″;

R₅ is an optionally substituted alkyl, an optionally substituted aryl, an optionally substituted heteroaryl, an optionally substituted aralkyl, or an optionally substituted heteroaralkyl;

R₆ is an optionally substituted aralkyl or an optionally substituted heteroaralkyl;

R′ for each occurrence, is H, —C(O)R′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R″ for each occurrence, is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl; and

R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl.

In still another aspect, the invention provides a method for treating neoplasia in a subject, the method involving administering to the subject an effective amount of a compound that is

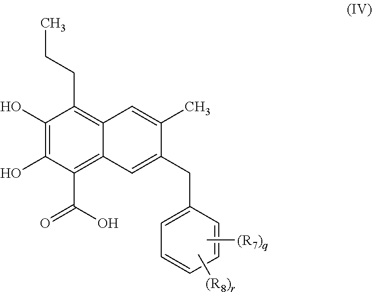

where,

each R₇ and R₈ is independently:

(i) an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

(ii) an optionally substituted haloalkyl, cyano, nitro, azido, or halo;

(iii) OR′, SR′, S(O)R′, S(O)₂R′, N(R′)₂, C(O)R′, C(S)R′, C(S)NR′R′, C(NR′)R′, C(NR′)NR′R′, C(O)NR′R′, C(O)NR′OR′, C(O)OR′, OC(O)R′, OC(O)OR′, NR′C(O)NR′R′, NR′C(S)NR′R′, NR′C(O)R′, NR′C(O)OR′, OC(O)NR′R′, or S(O)ᵣNR′R′; or

(iv) R₇ and R₈ may together with the carbon atoms to which each is attached, form a fussed bicyclic aryl, heteroaryl, cycloalkyl, or heterocycloalkyl, each of which may be optionally substituted;

R′ for each occurrence, is H, —C(O)R′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

q is 0, 1, 2, or 3; and

r is 0, 1, or 2.

In various embodiments of any of the above aspects, the compound is

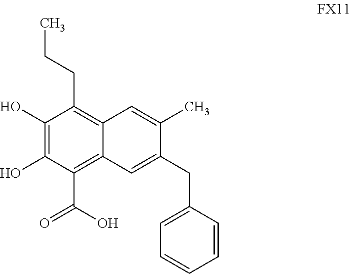

or an analog thereof.

In still another aspect, the invention provides a method for treating neoplasia in a subject, the method involving administering to the subject an effective amount of an agent that selectively inhibits lactate dehydrogenase A activity, thereby treating the neoplasia.

In still another aspect, the invention provides a method for treating neoplasia in a subject, the method involving administering to the subject an agent that competitively inhibits the conversion of pyruvate to lactate by lactate dehydrogenase A, thereby treating the neoplasia.

In still another aspect, the invention provides a method for treating neoplasia in a subject, the method involving administering to the subject an agent that selectively binds lactate dehydrogenase A, thereby treating the neoplasia.

In various embodiments of the above aspects, the agent is a compound of any one of Formulas I-IV, and tautomers, stereoisomers, Z and E isomers, optical isomers, N-oxides, hydrates, polymorphs, pharmaceutically acceptable esters, salts, prodrugs and/or isotopic derivatives thereof. In one example, the compound is FX11. In other embodiments of the above aspects, the neoplasia is characterized as having increased glycolytic metabolism relative to a control cell. In still other embodiments of the above aspects, the neoplasia is a solid tumor or hematological malignancy. In other embodiments of the above aspects, the neoplasia is selected from the group consisting of a lymphoma, B lymphoma, leukemia, brain cancer, colon cancer, glioblastoma, medulloblastoma, breast cancer, and pancreatic cancer. In other embodiments of the above aspects, the neoplasm is characterized as PET-positive. In other embodiments of the above aspects, the method further comprises administering an effective amount of NAD⁺ synthesis inhibitor FK866.

In another aspect, the invention provides a method for treating a subject having a neoplasm, the method involving administering to the subject a pharmaceutical composition containing an effective amount of an agent that reduces the expression or activity of lactate dehydrogenase A and an NAD⁺ synthesis inhibitor. In other embodiments of the above aspects, the NAD⁺ synthesis inhibitor is FK866. In other embodiments of the above aspects, the agent is an inhibitory nucleic acid molecule, a compound of Formula I-IV, FX11, or an analog or derivative thereof. In other embodiments of the above aspects, the inhibitory nucleic acid molecule is an siRNA that targets an LDHA sequence selected from the group consisting of, sequence 1 GGAGAAAGCCGUCUUAAUU; sequence 2, GGCAAAGACUAUAAUGUAA; sequence 3, UAAGGGUCUUUACGGAAUA; sequence 4, AAAGUCUUCUGAUGUCAUA. In another embodiment, two, three, or four of the siRNAs are provided.

In another aspect, the invention provides a method for selecting a therapeutic regimen for a subject identified as having a neoplasia, the method involving characterizing a neoplasia as having increased glycolysis relative to a control, where the increase indicates that the subject should be treated with a lactate dehydrogenase A inhibitor. In another embodiment, the method further indicates that an NAD⁺ synthesis inhibitor (e.g., FK866) should also be administered. In one embodiment, the increase in glycolysis is detected in a PET scan. In another embodiment, the increase is documented in a form for display (e.g., paper, computer screen).

In yet another aspect, the invention provides a composition for detecting a neoplasia having increased glycolytic metabolism, the composition containing a compound of any of Formulas I-IV containing a detectable moiety (e.g., radionuclide).

In a related aspect, the invention provides a composition for detecting a neoplasia having increased glycolytic metabolism, the composition containing FX11 conjugated to a detectable moiety. In one embodiment, the detectable moiety is conjugated at R4 of Formula I. In another embodiment, the detectable moiety comprises a radionuclide (e.g., a positron emitter or a gamma emitter). In another embodiment, the detectable moiety is detected using PET or SPECT imaging.

In yet another aspect, the invention provides a method for diagnosing a subject as having a neoplasia having increased glycolytic metabolism, the method involving contacting the subject with an effective amount of a composition of any of the above aspects containing a detectable moiety, and imaging the neoplasia (e.g., by PET or SPECT scan). In one embodiment, the method further comprises displaying the image in a readable form.

In still another aspect, the invention provides a kit for the treatment of a neoplasia, the kit containing an effective amount of an agent that reduces the expression or activity of lactate dehydrogenase A and directions for the use of the kit for the treatment of a neoplasia. In one embodiment, the kit further comprises an NAD⁺ synthesis inhibitor. In another embodiment, the agent is a lactate dehydrogenase A inhibitor that is a compound of Formula I-IV, FX11, or an analog or derivative thereof, or an LDHA inhibitory nucleic acid molecule.

In yet another aspect, the invention provides a kit for the diagnosis or characterization of a neoplasia, the kit containing an lactate dehydrogenase A inhibitor containing a detectable moiety and directions for the use of the kit for the diagnosis or characterization of a neoplasia. In one embodiment, the lactate dehydrogenase A inhibitor is a compound of Formula I conjugated to a detectable moiety at R4.

In yet another aspect, the invention provides a method for identifying an agent for the treatment of a glycolytic neoplasia, the method involving contacting a neoplastic cell that expresses lactate dehydrogenase A with a candidate compound (e.g., a derivative of FX11 or E); and identifying a decrease in lactate dehydrogenase A activity, thereby identifying the agent as useful in the treatment or prevention of a glycolytic neoplasia. In one embodiment, the compound is a compound of any of Formulas I-IV. In another embodiment, the compound is FX11, or an analog or derivative thereof. In yet another embodiment, the method further comprises detecting an increase in cell death, or a reduction or stabilization of neoplastic cell proliferation. In one embodiment, the neoplastic cell is a mammalian cell in vivo or in vitro.

In various embodiments of any of the above aspects, the subject has an end-stage neoplasm. In still other embodiments, the subject is identified as having a PET positive neoplasm or having a neoplasia having increased glycolytic metabolism relative to a reference. In other embodiments of any of the above aspects, the agent is administered locally via catheter or systemically. In other embodiments of any of the above aspects, the subject is a human identified as having a neoplasia having increased glycolysis relative to a reference. In other embodiments of any of the above aspects, the agent is administered at about 42-75 (e.g., 40, 45, 50, 55, 60, 65, 70, and 75) mg/kg/day or 75-200 (e.g., 75, 80, 85, 100, 110, 120, 130, 140, 150, 160, 170, 180, 190, and 200) mg/kg/day. In other embodiments of any of the above aspects, the agent is administered at about 100, 120, or 150 mg/kg/day.

The invention provides compositions for the diagnosis or treatment of neoplasias, including lymphomas, leukemias, brain cancers (e.g., glioblastomas, medulloblastomas), breast cancer, colon cancer, and pancreatic cancer. Compositions and articles defined by the invention were isolated or otherwise manufactured in connection with the examples provided below. Other features and advantages of the invention will be apparent from the detailed description, and from the claims.

### DEFINITIONS

By “lactate dehydrogenase A” is meant an enzyme that is predominantly expressed in muscle that converts pyruvate to lactate, a polypeptide having at least about 85% sequence identity to NCBI Accession No. NP_{—}005557 or NP_{—}001128711. Exemplary sequences are provided below:

```
NP_005557 L-lactate dehydrogenase A isoform 1
    1 matlkdqliy nllkeeqtpq nkitvvgvga vgmacaisil mkdladelal vdviedklkg
  61 emmdlqhgsl flrtpkivsg kdynvtansk lviitagarq qegesrlnlv qrnvnifkfi
121 ipnvvkyspn ckllivsnpv diltyvawki sgfpknrvig sgcnldsarf rylmgerlgv
181 hplschgwvl gehgdssvpv wsgmnvagvs lktlhpdlgt dkdkeqwkev hkqvvesaye
241 viklkgytsw aiglsvadla esimknlrrv hpvstmikgl ygikddvfls vpcilgqngi
301 sdlvkvtlts eeearlkksa dtlwgiqkel qf;
and
NP_001128711 L-lactate dehydrogenase A isoform 2
    1 matlkdqliy nllkeeqtpq nkitvvgvga vgmacaisil mkdladelal vdviedklkg
  61 emmdlqhgsl flrtpkivsg kvdiltyvaw kisgfpknrv igsgcnldsa rfrylmgerl
121 gvhplschgw vlgehgdssv pvwsgmnvag vslktlhpdl gtdkdkeqwk evhkqvvesa
181 yeviklkgyt swaiglsvad laesimknlr rvhpvstmik glygikddvf lsvpcilgqn
241 gisdlvkvtl tseeearlkk sadtlwgiqk elqf
```

By “lactate dehydrogenase A activity” is meant the conversion of pyruvate to lactate, a cell proliferative activity, or any other enzymatic activity of lactate dehydrogenase A, or a fragment thereof. A schematic diagram illustrating this pathway is shown in FIG. 4.

By “lactate dehydrogenase A inhibitory nucleic acid molecule” is meant an siRNA, antisense oligonucleotide, or shRNA that binds a lactate dehydrogenase A nucleic acid sequence and reduces the expression of lactate dehydrogenase A.

By “lactate dehydrogenase A nucleic acid molecule” is meant a polynucleotide that encodes lactate dehydrogenase A.

By “lactate dehydrogenase A inhibitor” is meant any agent that reduces the conversion of pyruvate to lactate by lactate dehydrogenase A, that reduces a lactate dehydrogenase A proliferative activity or that otherwise reduces a lactate dehydrogenase A enzymatic activity. Such reduction need not be complete but is preferably detectable. For example, a reduction by at least about 10%, 20%, 30%, 40%, 50%, 75%, 80%, or even by as much as 90%, 95% or more. In one embodiment, an agent of the invention competitively inhibits the conversion of pyruvate to lactate. In another embodiment, a lactate dehydrogenase A inhibitor “selectively inhibits” an enzymatic activity of lactate dehydrogenase A. Such inhibition is “selective” so long as the agent inhibits lactate dehydrogenase A to a greater extent than the agent inhibits lactate dehydrogenase B.

By “glycolytic metabolism” is meant cellular energy production from glucose. A neoplastic cell characterized as having a “glycolytic metabolism” need not rely exclusively on glycolysis, but will show increased glycolysis relative to a corresponding control cell. Preferably, the increase is significant and/or detectable. For example, an increase of at least about 10%, 20%, 30%, 40%, 50%, 75%, 80%, or even by as much as 90%, 95% or more.

By “readable form” is meant a medium for display of information. Information in a readable form may be displayed on paper, on a computer screen, or in any other concrete format that provides for communication of the information.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change, preferably a 25% change, more preferably a 40% change, and most preferably a 50% or greater change in expression or activity.

By “analog” is meant a molecule that is not identical, but has analogous functional or structural features.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable moiety” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “reference” is meant a standard or control condition. In one embodiment, the activity of lactate dehydrogenase A (LDHA) in a neoplastic cell is compared to the activity of LDHA in a reference, such as a control cell obtained from a corresponding tissue.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

### BRIEF DESCRIPTION OF THE DRAWINGS

FIGS. 1A-1C are diagrams showing an analysis of human cancer gene expression profiles through the Broad Institute GSEA site reveals that leukemias, lymphomas, and brain cancers have high glycolytic gene expression.

FIG. 2 is a diagram showing an analysis of human cancer gene expression profiles through the Broad Institute GSEA site reveals that leukemias and pancreatic cancers have the oxidative phosphorylation signature suggestive of glutamine utilization and glutaminolysis in these and other cancers.

FIG. 3 is a graph showing an analysis of enzyme gene mutations from the Hopkins (Jones et al. Science. 2008; 321(5897):1801-6; Parsons et al. Science. 2008; 321(5897):1807-12; Wood et al. Science. 2007; 318(5853):1108-13. Sjoblom et al. Science. 2006; 314(5797):268-74.) and CGA (Cancer Genome Atlas Research Network. Nature. 2008; 455(7216):1061-8.) dataset reveals that metabolic enzyme mutations (particularly in the glycolytic, TCA cycle, and respiratory chain) are prevalent in brain cancers.

FIG. 4 is a schematic diagram showing the pathway by which lactate dehydrogenase A (LDHA) converts pyruvate to lactate with the concomitant production of NAD+. FX11 and E (FX11; 2,3-dihydroxy-6-methyl-7-(phenylmethyl)-4-propylnaphthalene-1-carboxylic acid, Pubchem ID: 10498042) and 11e (E; 2,3-dihydroxy-6-methyl-7-(methyl)-4-propylnaphthalene-1-carboxylic acid, Pubchem ID: 10265351) are related compounds that inhibit LDHA in vitro (Deck et Selective inhibitors of human lactate dehydrogenases and lactate dehydrogenase from the malarial parasite Plasmodium falciparum. J Med. Chem. 1998; 41(20):3879-87)

FIG. 5 provides two graphs showing a short interference RNA (siRNA) reduction of LDHA (see immunoblot on the right of each growth curves graph) as compared to siControl diminishes the growth of a human lymphoma model cell line P493 and human prostate cancer cell line P198.

FIGS. 6A-6C show that a reduction of LDHA expression by siRNA leads to increased oxygen consumption and oxidative stress-induced cell death of P493 human lymphoma B cells. siRNAs targeting human LDHA (SMARTpool) was transfected via electroporation to transiently knock-down the LDHA expression. FIG. 6A is a graph showing oxygen consumption of P493 cells, which was determined by the use of Clark-type oxygen electrode at 72 hour post-transfection with siLDHA or siControl. FIG. 6B is an immunoblot, which was performed on whole-cell lysates and probed with rabbit monoclonal anti-LDHA and re-probed with anti-α-tubulin as a loading control. FIG. 6C is a graph showing intracellular ROS production detected with DCFDA fluorescence and monitored by flow cytometry at 72 hour post-transfection with siLDHA or siControl in the presence or absence of N-acetylcysteine (NAC). FIG. 6D shows the results of a FACS analysis of cell death using Annexin V and 7-AAD stained cells at 96 hour post-transfection with siLDHA or siControl in the presence or absence of NAC. FIG. 6E is a graph showing cell population growth of siControl cells compared with cells treated with siLDHA grown in the presence or absence of 20 mM NAC given 24 hour post-transfection.

FIGS. 7A-7C are graphs. FIGS. 7A and 7B show that reduction of LDHA by siRNA increased oxygen consumption in P198 human pancreatic cancer cells. Oxygen consumption was determined by the use of Clark-type oxygen electrode. The immunoblot was performed on whole-cell lysates and probed with rabbit monoclonal anti-LDHA antibody and re-probed with anti-α-tubulin as a loading control. Data are shown as mean±SD. FIG. 7C is a graph showing that FX11 does not affect c-Myc levels in P493 cells. Western blot analysis was performed after 24 hours of treatment with 30 μM FX11. Equivalent amounts of proteins were immunoblotted with anti-c-myc antibody and α-tubulin served as a loading control. Tetracycline, which is known to repress c-Myc expression, was used as a control.

FIGS. 8A-8D are graphs. FIGS. 8A and B show that FX11 and its derivative E are competitive inhibitors of LDHA with NADH as substrate. Lineweaver-Burk plots were determined from triplicate experiments using averages of activities. Ki determination was performed with 13.5 μM FX11 or 27 μM E. FIG. 8C is a graph showing affinity chromatography of LDHA using Sepharose-immobilized FX11 or E. Equal volumes of P493 human B cell lysates were chromatographed with 6 column volumes of high salt (1 M NaCl) wash followed by elution with 1 mM NADH. LDHA activity was determined for each fraction, and the experiment was replicated with a representative experiment shown. FIG. 8D shows the results of an assay of FX11 LDHA inhibitory activity.

FIGS. 9A-9E show that inhibition of LDHA by FX11 resulted in increased oxygen consumption, ROS production and cell death. FIG. 9A is a graph showing oxygen consumption of P493 cells, which was determined by a Clark-type oxygen electrode in the presence and absence of FX11. Data are representative of duplicate experiments. FIG. 9B is a graph showing reactive oxygen species (ROS) levels, which were determined by DCFDA fluorescence in P493 cells treated with FX11 or FK866. Data are representative of triplicate samples of two separate experiments. FIG. 9C show results of a FACS analysis. Cell death was determined by flow cytometry of Annexin V and 7-AAD stained cells after 24 hours of FX11 treatment as compared to control. FIGS. 9D and 9E are graphs showing cell population growth of control cells compared with cells treated with FX11 or FK866 in the presence or absence of 20 mM NAC. All cells were grown at 1×10⁵ cells/ml. Cell counts were done in triplicate and shown as mean±SD and the entire experiment was replicated with similar results.

FIGS. 10A-10F show the effects of FX-11 and FK866 on cultured cells. FIG. 10A is a FACS analysis showing that FK866 enhanced FX11-induced a loss of mitochondrial membrane potential. P493 cells treated with control vehicle, FK866, FX11 or with both inhibitors were stained with JC-1 and subjected to flow cytometric analysis with FL2 representing red fluorescence and FL1 representing green fluorescence intensity, which is reflective of cells with decreased mitochondrial membrane. The percentage of cells with decreased membrane potential is indicated in each panel. A duplicate experiment yielded similar results. FIG. 10B is a graph showing that FK866 enhanced FX11-mediated inhibition of cell proliferation. Live cells were counted using trypan blue dye exclusion. Data are shown as mean±SD of triplicate samples. FIG. 10C is a graph showing that FX11 or FK866 decreases ATP levels. P493 cells were treated with 9 μM FX11 or 0.5 nM FK866 for 20 hours and counted. ATP levels were determined by luciferin-luciferase-based assay on aliquots containing equal number of live cells. (*): p=0.0004, (**): p=0.004. FIG. 10D shows an immunoblot of phosphor-AMPK in lysates of cell treated with FX11 or FK866. Tubulin serves as a loading control. AICAR, an AMP analog that activates AMPK, was used to treat the cells as a positive control. FIG. 10E is a graph showing that FX11 increased the NADH/NAD⁺ ratio. NADH/NAD⁺ ratio in P493 cells treated with 9 μM FX11 for 24 hours as compared with vehicle control. (*): p=0.028. FIG. 10F shows that FX11 inhibited lactate production. Lactate levels in the media of P493 human B cells treated with 9 μM FX11 or 0.5 nM FK866 for 24 hours as compared with control. Control RPMI contained 10.7 mmol/L glucose and no detectable lactate. (*): p=6.9E-06.

FIGS. 11A, B, C, D, E, F and 11G are graphs showing the results of treating various neoplastic cells with FX11 in vitro. RCC4 cells and MCF-7 cells were more sensitive to FX11 than RCC4-VHL and MDA-MB-453 cells. FIGS. 11A-11D show that FX11 inhibited cell population growth of human renal carcinoma RCC4 and RCC4-VHL cells or human breast cancer MCF-7 and MDA-453 cells when administered at an effective dosage. FIG. 11E shows that the effect of FX11 on the proliferation of P493 cells was glucose-dependent. FIG. 11F is a graph showing the LDHA-dependent effect of FX11. Cell population growth of P493 cells with siRNA-mediated reduced LDHA expression in the presence or absence of 9 μM FX11. FIG. 11G shows that Ramos Burkitt lymphoma cells are sensitive to FX11 inhibition in a manner that is diminished by glucose withdrawal, which caused a decrease in cell proliferation.

FIGS. 12A and 12B are graphs showing characterization of glucose, glutamine and pyruvate dependency of different human breast cancer cell lines, MCF-7 and MDA-MB-453. Cells were cultured in media with glucose, glutamine or pyruvate. All media were supplemented with 10% bovine fetal serum and 1% penicillin-streptomycin. Averages of cell numbers from triplicate experiments are shown ±SD.

FIGS. 13A-13D are graphs show the effect of hypoxia on cells treated with FX11. FIGS. 13A and 13B show that hypoxia accentuated the sensitivity of human P493 B cells. FIGS. 13C and 13D show that hypoxia also accentuated the sensitivity of human P198 pancreatic cancer cells to FX11 inhibition of growth. Cell population growth of P493 B cells or pancreatic cancer P198 cells in normoxia or hypoxia with different doses of FX11. Normoxic cells were grown at 37° C. in a 5% CO₂, 95% air incubator. Hypoxic cells (1% O₂) were maintained for the indicated time in a controlled atmosphere chamber with a gas mixture containing 1% O₂, 5% CO₂, and 94% N₂ at 37° C. There is no significant difference in cell numbers between 0% and 0.1% DMSO groups. FIG. 13E shows that FX11 inhibited human Ramos Burkitt lymphoma cell population growth in a dose-dependent manner in normoxia and hypoxia. FIG. 13F shows that FX11 inhibits growth of human pancreatic cell lines E3LZ10.7 and P10. FIG. 13G shows that FX11 inhibits human glioblastoma U-87-MG cells in a dose-dependent manner. FIG. 13H shows that FX11 inhibits P493 proliferation under normoxic and hypoxic conditions.

FIGS. 14A-14D show the in vivo efficacy of FX11 as an anti-tumor agent. FIG. 14A is a micrograph showing that P493 lymphoma hypoxic regions were detected with pimonidazole staining (red) followed by immunofluorescent microscopy. FIG. 14B shows the effect of FX11 on growth of palpable human P493 B cell xenografts. Control animals were treated with daily IP injection of vehicle (2% DMSO), and doxycycline (0.8 mg/day) was used as a positive control because it inhibits Myc expression and tumorigenesis in P493 cells. FIG. 14C shows the Effect of FX11 and/or FK866 daily treatment as compared with control or compound E (a weak LDHA inhibitor) on established human lymphoma xenografts. The inset in panel 14C shows photos of representative animals treated with control vehicle or FX11. FIG. 14D shows that FX11 inhibited P198 human pancreatic cancer xenografts as compared with E. For experiments in all panels, 2.0×10⁷ P493 cells or 5×10⁶ P198 cells were injected subcutaneously into SCID mice or athymic nu mice, respectively. When the tumor volume reached 200 mm³, 42 μg of FX11 and/or 100 μg of FK866 was injected intra-peritoneally daily and the tumors were observed for 10 to 14 days. The tumor volumes were measured using a digital caliper every 4 days and calculated using the following formula: [length (mm)×width (mm)×width (mm)×0.52]. The results represent the average ±SEM.

FIG. 15 shows that FX11 treatment for 24 hours resulted in the reduction of glucose utilization by P493 cells and lactate production in the media. Control RPMI has 10.7 mmol/L glucose and no detectable lactate as determine by the xxx glucose/lactate ABL700 Radiometer analyzer. In the absence of glutamine, FX11 diminishes lactate production while glucose consumption is still maintained (with a glucose consumption to lactate production molar ratio of 2:1 compared to the 1:2 ratio found in untreated control cells).

FIG. 16 is a schematic diagram showing how FK866 inhibits the first step of NAD synthesis.

FIG. 17 provides a series of graphs showing blood chemistries for animals treated with FX11 or FK866 alone or in combination at doses that affected tumor growth.

### DETAILED DESCRIPTION OF THE INVENTION

The invention features compositions and methods featuring lactate dehydrogenase A inhibitors that are useful for the treatment or prevention of a neoplasia (e.g., lymphoma, leukemia, brain cancer, glioblastoma, medulloblastoma, breast cancer, colon cancer, and pancreatic cancer), as well as imaging agents useful in diagnosing a neoplasia having increased glycolytic metabolism relative to a reference.

As the result of genetic alterations and tumor hypoxia, many cancer cells avidly take up glucose and generate lactate through lactate dehydrogenase A (LDHA), which is encoded by a target gene of c-Myc and HIF-1. The invention is based, at least in part, on the discovery that reducing LDHA by siRNA or with a small molecule (FX11) not only reduces ATP levels but also induces significant oxidative stress that triggers cell death. Furthermore, FX11, an inhibitor of human LDHA displayed remarkable activity in cultured tumor cells and inhibited established lymphoma and pancreatic tumor xenografts. The activity of FX11 was accentuated by another metabolic inhibitor, FK866 (APO866), which inhibited NAD+ synthesis through direct inhibition of nicotinamide phosphoribosyltransferase (NAMPT) (Hasmann and Schemainda, (2003). Cancer Res 63, 7436-7442; Nahimana et al., (2009) Blood 113, 3276-3286). Without wishing to be bound by theory, the sensitivity of cancer cells to these agents appears linked to their ability to induce oxidative stress through increased reactive oxygen species (ROS). The results reported herein indicate that targeting cancer metabolism through small molecules is achievable and hence paves the way for the development of new classes of anti-cancer drugs.

### Glycolysis and Neoplasia

Over 80 years ago, Otto Warburg described the propensity of cancer tissues and cells to take up glucose avidly and convert most of it to lactate, even under experimental conditions with adequate oxygen. Warburg postulated that cancer cells must have acquired defective mitochondria and hence rely on glycolysis for energy metabolism. This phenomenon, which has been termed the Warburg effect or aerobic glycolysis, is distinct from the process of anaerobic glycolysis that is activated in hypoxia. Although there was substantial interest in the Warburg effect in the 1960s to early 1980s, later findings that mitochondrial function was preserved in some cancers and the emergence of oncogenes and molecular biology led to a diminished interest in this effect. However, recent mechanistic understanding of tumorigenesis through the activation of oncogenes and loss of tumor suppressor genes renewed an interest in oncogenic alterations of metabolism in cancers, with a deeper mechanistic understanding. This understanding has been underpinned by the direct links between activation of oncogenes and loss of tumor suppressors to the regulation of cellular metabolism. Loss of the tumor suppressors VHL, fumarate hydratase (FH), and succinate dehydrogenase (SDH) have all been linked to the stabilization of the hypoxia inducible factor HIF-1, which is otherwise stabilized normally in hypoxic cells for cellular adaptation and survival. Remarkably, HIF-1 is also stabilized downstream of a mutant isocitrate dehydrogenase 1 (IDH1), which is found in over 80% of gliomas. HIF-1 is a critical transcription factor for the activation of glycolytic enzyme genes including lactate dehydrogenase A (LDHA), which converts pyruvate to lactate coupled with the recycling of NAD⁺.

Lactate dehydrogenase is a tetrameric enzyme comprising of two major subunits A and/or

B, resulting in five isozymes (A₄, A₃B₁, A₂B₂, A₁B₃, B₄). LDHA (LDH-5, M-LDH or A₄), which is the predominant form in skeletal muscle, favors the conversion of pyruvate to lactate. LDHB (LDH-1, H-LDH or B₄), which is found in heart muscle, converts lactate to pyruvate that is further oxidized. It has been long known that many human cancers have higher LDHA levels than normal tissues, but the link between oncogenes and glycolysis was poorly understood. Early studies documented, however, that overexpression of Src and Ras oncogenes in fibroblasts could activate aerobic glycolysis and that LDHA is among a few glycolytic enzymes which were tyrosine phosphorylated in Src-transformed cells (Cooper et al., 1984 J Biol Chem 259, 7835-7841; Dang and Semenza, 1999 Trends Biochem Sci 24, 68-72). LDHA was further identified as a direct target gene of the c-Myc oncogenic transcription factor (Lewis et al., 1997 Mol Cell Biol 17, 4967-4978; Shim et al., 1997 Proc Natl Acad Sci USA 94, 6658-6663). HIF also activates LDHA (Firth et al., 1995 J Biol Chem 270, 21021-21027; Semenza et al., 1996 J Biol Chem 271, 32529-32537), which uniquely resides at the crossroads of c-Myc and hypoxia.

Reduction of LDHA expression disables the metabolic adaptive response of human Burkitt lymphoma cell lines, and profoundly inhibits soft agar colony formation in both Burkitt cells and c-Myc-transformed Rat1a fibroblasts that have reduced LDHA expression through anti-sense RNA expression (Shim et al., 1997 Proc Natl Acad Sci USA 94, 6658-6663). Nine years later, Fantin et al. (Fantin et al., 2006 Cancer Cell 9, 425-434) used short hairpin RNAs (shRNAs) to reduce LDHA expression and inhibit mouse mammary tumorigenesis. More recently, a surrogate cell model of hereditary leiomyoma and renal cell carcinoma (HLRCC) was also found to display diminished tumorigenesis when LDHA was reduced by shRNAs (Xie et al., 2009 Mol Cancer Ther 8, 626-635). These studies provide proof-of-concept that LDHA is a tractable therapeutic target, particularly in light of the fact that humans lacking LDHA are essentially normal, except for exertional myoglobinuria upon exercising (Maekawa et al., 1986 μm J Hum Genet. 39, 232-238). Although these studies suggested that reducing LDHA levels could prevent the formation of tumors, the previous work failed to show that reducing LDHA levels or activity could be used to treat an established tumor.

### Neoplastic Disease Therapy

Methods of this invention are suitable for administration to humans with neoplastic diseases, particularly those neoplasias identified as having a glycolytic metabolism. Such neoplasias are identified, for example, using PET imaging. A PET positive neoplasia is identified as amenable to treatment using the methods of the invention. The methods comprise administering an amount of a pharmaceutical composition containing a LDHA inhibitor (e.g., a compound of Formula I-IV, FX11, a derivative thereof) in an amount effective to decrease a biological activity of LDHA, such as lactate dehydrogenase enzyme activity, to achieve a desired effect, be it palliation of an existing tumor mass or prevention of recurrence. The LDHA inhibitor (e.g., e.g., a compound of Formula I-IV, FX11, a derivative thereof) is useful alone or in combination with a nicotinamide phosphoribosyltransferase (NAMPT) inhibitor of NAD⁺ synthesis (e.g., FK 866).

A tumor comprises one or more neoplastic cells, or a mass of neoplastic cells, and can also encompass cells that support the growth and/or propagation of a cancer cell, such as vasculature and/or stroma. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

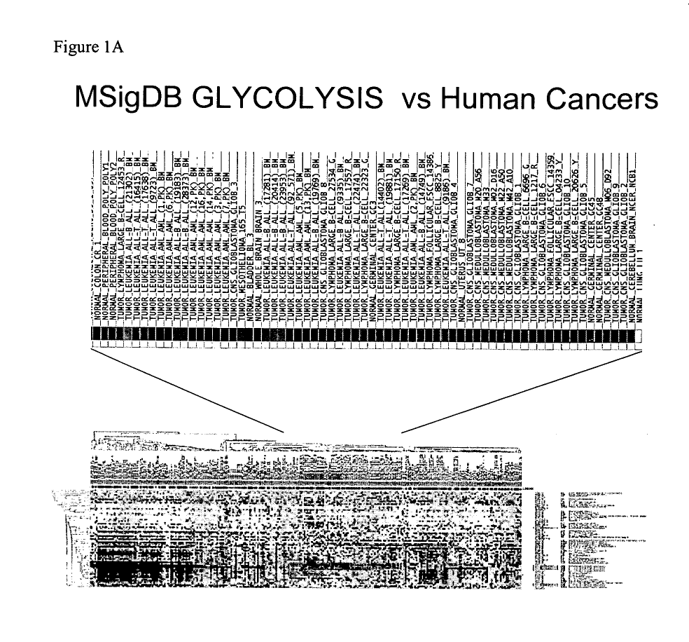

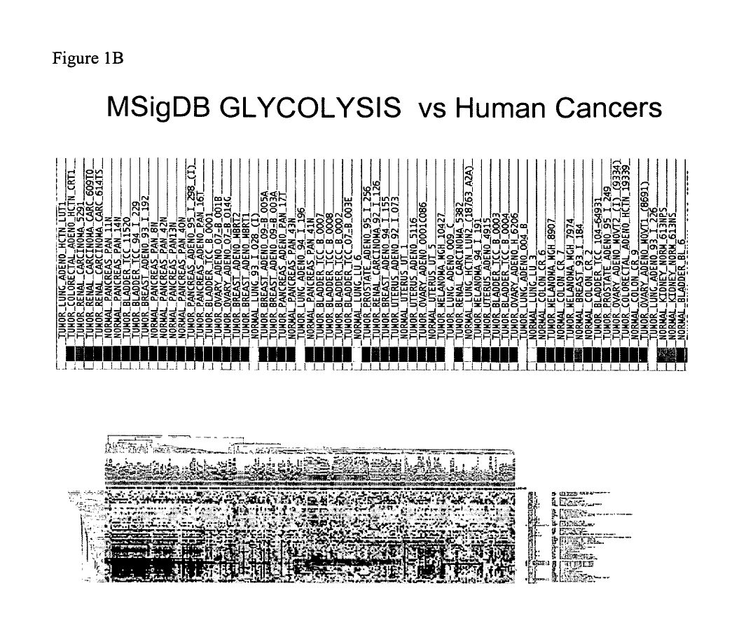

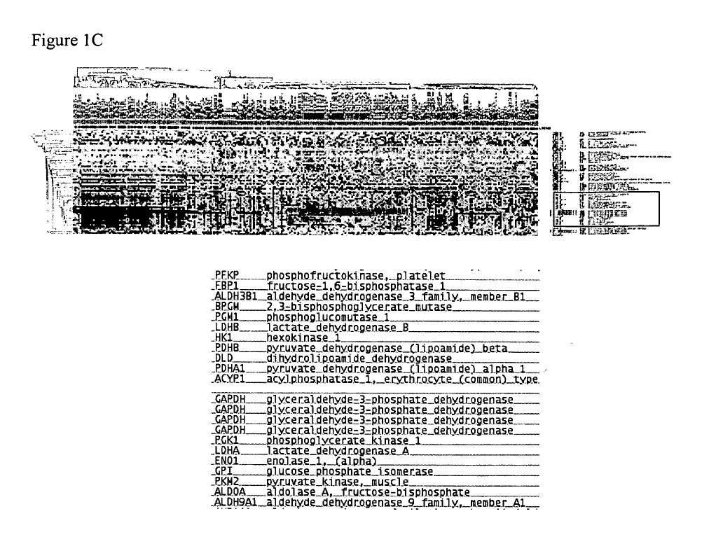

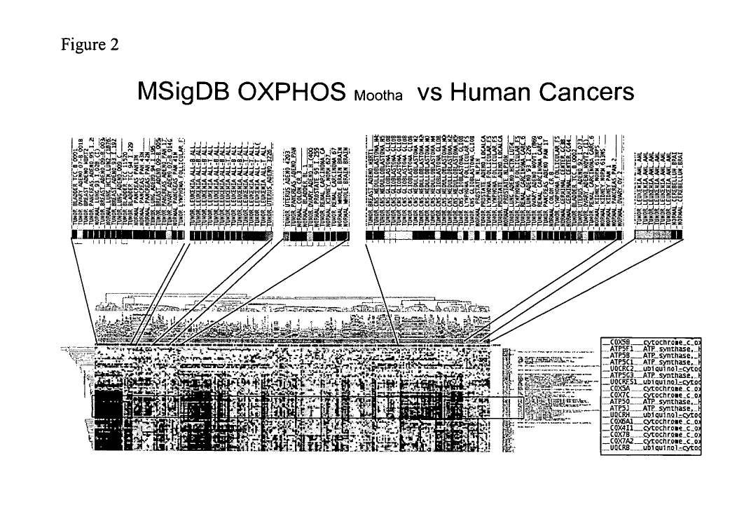

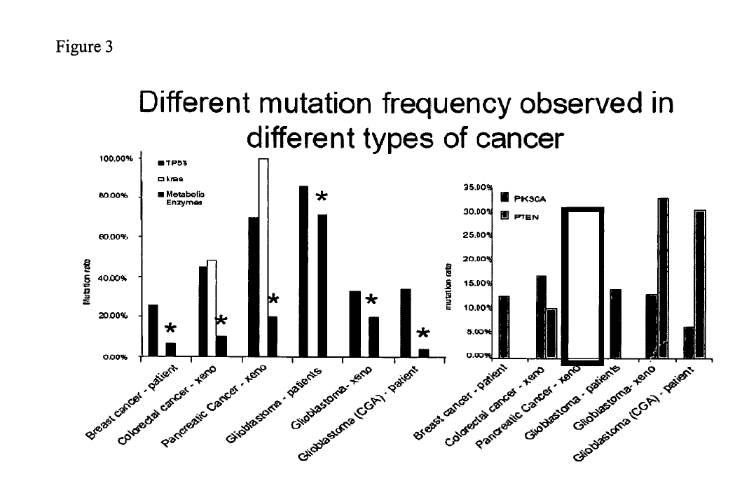

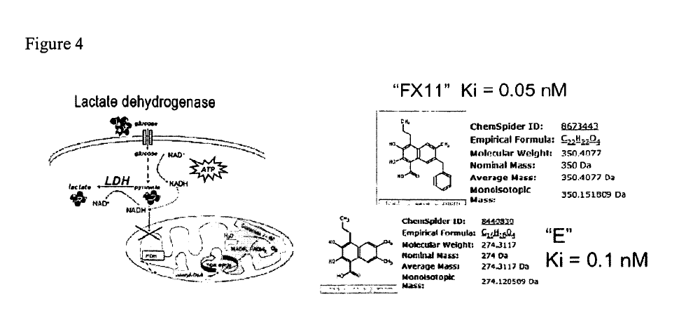

The present invention includes compositions and methods for reducing the growth and/or proliferation of a neoplastic cell, particularly a neoplastic cell having an altered metabolic profile, such as a neoplasia that utilizes glycolysis or glutaminolysis. In particular embodiments, the invention provides for the treatment of a neoplasia having a mutation that increases glycolysis or glutaminolysis. Mutations indicative of an increase in glycolysis include alterations in PIK3CA, loss of PTEN, or activation of AKT, MYC or HIF. Examples of neoplasias having high glycolytic gene expression are shown in FIG. 1, and include leukemias and brain cancers (e.g., glioblastoma, medulloblastoma). Alterations in oxidative phosphorylation are indicative of glutaminolysis (FIG. 2). Neoplasias having such alterations include leukemias and pancreatic cancers. Metabolic alterations include mutations in metabolic enzymes involved in glycolysis, the tricarboxylic acid cycle, and the respiratory chain. Metabolic alterations are common in brain cancers (e.g., glioblastoma) (FIG. 3).

### Selection of a Treatment Method

After a subject is diagnosed as having a neoplasia, a method of treatment is selected. Subjects having a neoplasia associated with alterations in metabolism, such as an increase in glycolytic metabolism, are identified as amenable to treatment with a composition or method of the invention. Such subjects can be identified using any method known in the art. In one approach, a subject is identified by scanning to identify the presence or absence of an increase in glycolytic metabolism in a neoplasia (e.g., a tumor) within the subject, for example, using positron emission tomography. Subjects having tumors that can be visualized, i.e., PET-positive tumors, are identified as amenable to treatment with a method of the invention.

### Compounds of the Invention

The LDHA inhibitor FX11 was found to inhibit neoplasias characterized by a glycolytic metabolism, including lymphomas, leukemias, brain cancers (e.g., glioblastomas, medulloblastomas), breast cancer, colon cancer, and pancreatic cancer. Accordingly, the invention provides methods of treating neoplasia featuring compounds of Formula I:

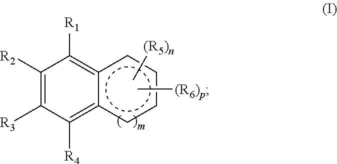

wherein,

R₁ is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, an optionally substituted heteroaralkyl, —C(O)R′, —OR″, —S(O)ₘR′, —NR′R″, or haloalkyl;

R₂ is H, —C(O)R′, —OR″, —S(O)ₘR′, —NR′R″, nitro, cyano, halogen, or haloalkyl;

R₃ is H, —C(O)R′, —OR″, —S(O)ₘR′, —NR′R″, nitro, cyano, halogen, or haloalkyl;

R₄ is H, an optionally substituted alkyl, an optionally substituted aryl, an optionally substituted heteroaryl, an optionally substituted aralkyl, an optionally substituted heteroaralkyl, —C(O)R′, —OR″, —S(O)ₘR′, or —NR′R″;

each R₅ is independently an optionally substituted alkyl, an optionally substituted aryl, an optionally substituted heteroaryl, an optionally substituted aralkyl, or an optionally substituted heteroaralkyl;

each R₆ is independently H, an optionally substituted alkyl, an optionally substituted aryl, an optionally substituted heteroaryl, an optionally substituted aralkyl, or an optionally substituted heteroaralkyl;

R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R″ for each occurrence, is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

m is 0, 1, or 2;

n is 1 or 2; and

p is 1 or 2.

In other embodiments, the invention provides methods of treating neoplasia featuring compounds of Formula II:

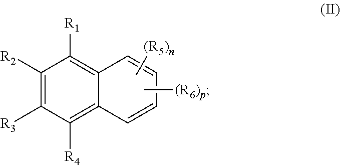

wherein,

R₁ is an optionally substituted alkyl, an optionally substituted aryl, an optionally substituted heteroaryl, an optionally substituted aralkyl, or an optionally substituted heteroaralkyl;

R₂ is H, —C(O)R′, —OR″, —NR′R″, halogen, or haloalkyl;

R₃ is H, —C(O)R′, —OR″, —NR′R″, halogen, or haloalkyl;

R₄ is —C(O)R′, —OR″, —S(O)ₘR′, or —NR′R″;

each R₅ is independently an optionally substituted alkyl, an optionally substituted aryl, an optionally substituted heteroaryl, an optionally substituted aralkyl, or an optionally substituted heteroaralkyl;

each R₆ is independently H, an optionally substituted alkyl, an optionally substituted aryl, an optionally substituted heteroaryl, an optionally substituted aralkyl, or an optionally substituted heteroaralkyl;

R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R″ for each occurrence, is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

n is 1 or 2; and

p is 1 or 2.

In yet another embodiment, the invention provides methods of treating a neoplasia featuring compositions of Formula III.

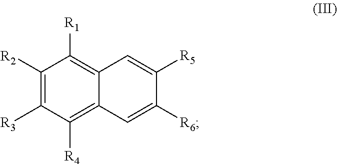

wherein,

R₁ is an optionally substituted alkyl or an optionally substituted aralkyl;

R₂ is H, —C(O)R′, —OR″, or —NR′R″;

R₃ is H, —C(O)R′, —OR″, or —NR′R″;

R₄ is —C(O)R′ or —OR″;

R₅ is an optionally substituted alkyl, an optionally substituted aryl, an optionally substituted heteroaryl, an optionally substituted aralkyl, or an optionally substituted heteroaralkyl;

R₆ is an optionally substituted alkyl, an optionally substituted aryl, an optionally substituted heteroaryl, an optionally substituted aralkyl, or an optionally substituted heteroaralkyl;

R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R″ for each occurrence, is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl; and

R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl.

In one preferred embodiment, the invention provides novel derivatives of FX11, wherein said derivatives comprise a hydrophobic moiety at the R6 position, which as reported below is important for LDHA inhibitory activity. Accordingly, the invention provides compounds of Formulas III and IV:

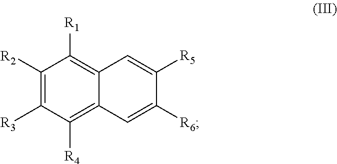

wherein,

R₁ is an optionally substituted alkyl or an optionally substituted aralkyl;

R₂ is H, —C(O)R′, —OR″, or —NR′R″;

R₃ is H, —C(O)R′, —OR″, or —NR′R″;

R₄ is —C(O)R′ or —OR″;

R₅ is an optionally substituted alkyl, an optionally substituted aryl, an optionally substituted heteroaryl, an optionally substituted aralkyl, or an optionally substituted heteroaralkyl;

R₆ is an optionally substituted aralkyl or an optionally substituted heteroaralkyl;

R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R″ for each occurrence, is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl; and

R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl.

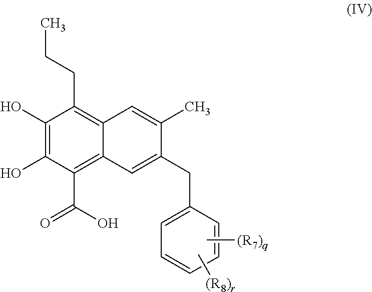

wherein,

each R₇ and R₈ is independently:

(i) an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

(ii) an optionally substituted haloalkyl, cyano, nitro, azido, or halo;

(iii) OR′, SR′, S(O)R′, S(O)₂R′, N(R′)₂, C(O)R′, C(S)R′, C(S)NR′R′, C(NR′)R′, C(NR′)NR′R′, C(O)NR′R′, C(O)NR′OR′, C(O)OR′, OC(O)R′, OC(O)OR′, NR′C(O)NR′R′, NR′C(S)NR′R′, NR′C(O)R′, NR′C(O)OR′, OC(O)NR′R′, or S(O)ᵣNR′R′; or

(iv) R₇ and R₈ may together with the carbon atoms to which each is attached, form a fussed bicyclic aryl, heteroaryl, cycloalkyl, or heterocycloalkyl, each of which may be optionally substituted;

R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;

q is 0, 1, 2, or 3; and

r is 0, 1, or 2.

The structure of FX11 is provided below:

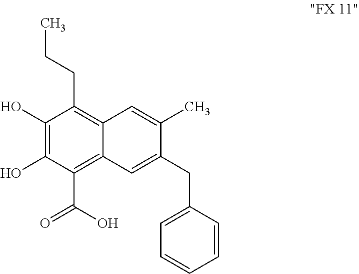

The invention further provides imaging reagents having a detectable moiety conjugated to the FX11 carboxyl group.

The structure of E is provided below:

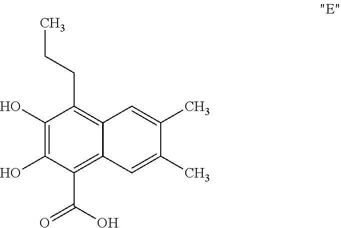

The invention further provides for derivatives of FX11 and E, which are screened to identify those having LDHA inhibitory activity. In particular embodiments, E derivatives will be tested to determine structure-activity relationships by modifying a side chain of E, as shown in the exemplary E-derivative structure provided below.

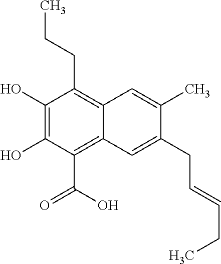

Derivatives of FX11 and/or E that inhibit LDHA and that reduce or stabilize the growth or proliferation of a neoplastic cell are selected as useful in the methods of the invention. If desired, such derivatives are also screened for activity against neoplasias in vivo (e.g., in mouse xenografts).

FX11 may be used alone, or in combination with a nicotinamide phosphoribosyltransferase (NAMPT) inhibitor of NAD⁺ synthesis (e.g., FK 866).

Without wishing to be bound by theory, FX11, analogs and derivatives thereof are particularly effective in inhibiting the proliferation or survival of a neoplasia. In certain embodiments, a compound of the invention can prevent, inhibit, or disrupt, or reduce by at least 10%, 25%, 50%, 75%, or 100% LDHA activity or LDHA expression.

In certain embodiments, a compound of the invention is a small molecule having a molecular weight less than about 1000 daltons, less than 800, less than 600, less than 500, less than 400, or less than about 300 daltons. Examples of compounds of the invention include compounds of Formula I, II, III, IV, and pharmaceutically acceptable salts thereof.

The term “pharmaceutically acceptable salt” also refers to a salt prepared from a compound of the invention having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)-amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)-amine, or tri-(2-hydroxyethyl)amine; N-methyl-D-glucamine; and amino acids such as arginine, lysine, and the like, or any other compound delineated herein, having a basic functional group, such as an amino functional group, and a pharmaceutically acceptable inorganic or organic acid. Suitable acids include, but are not limited to, hydrogen sulfate, citric acid, acetic acid, oxalic acid, hydrochloric acid, hydrogen bromide, hydrogen iodide, nitric acid, phosphoric acid, isonicotinic acid, lactic acid, salicylic acid, tartaric acid, ascorbic acid, succinic acid, maleic acid, besylic acid, fumaric acid, gluconic acid, glucaronic acid, saccharic acid, formic acid, benzoic acid, glutamic acid, methanesulfonic acid, ethanesulfonic acid, benzenesulfonic acid, and p-toluenesulfonic acid.

As used herein, the term “alkyl” refers to a straight-chained or branched hydrocarbon group containing 1 to 12 carbon atoms. The term “lower alkyl” refers to a C1-C6 alkyl chain. Examples of alkyl groups include methyl, ethyl, n-propyl, isopropyl, tert-butyl, and n-pentyl. Alkyl groups may be optionally substituted with one or more substituents.

The term “alkenyl” refers to an unsaturated hydrocarbon chain that may be a straight chain or branched chain, containing 2 to 12 carbon atoms and at least one carbon-carbon double bond. Alkenyl groups may be optionally substituted with one or more substituents.

The term “alkynyl” refers to an unsaturated hydrocarbon chain that may be a straight chain or branched chain, containing the 2 to 12 carbon atoms and at least one carbon-carbon triple bond. Alkynyl groups may be optionally substituted with one or more substituents.

The sp² or sp carbons of an alkenyl group and an alkynyl group, respectively, may optionally be the point of attachment of the alkenyl or alkynyl groups.

The term “alkoxy” refers to an —O-alkyl radical.

As used herein, the term “halogen”, “hal” or “halo” means —F, —Cl, —Br or —I.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

The term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon (with appropriate hydrogen atoms unless otherwise indicated). Heteroaryl groups may be optionally substituted with one or more substituents. In one embodiment, 0, 1, 2, 3, or 4 atoms of each ring of a heteroaryl group may be substituted by a substituent. Examples of heteroaryl groups include pyridyl, furanyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, isoquinolinyl, indazolyl, and the like.

The term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, tetrahydropyranyl, morpholinyl, thiomorpholinyl, 1,3-dioxolane, tetrahydrofuranyl, tetrahydrothienyl, thiirenyl, and the like.

The term “alkylamino” refers to an amino substituent which is further substituted with one or two alkyl groups. The term “aminoalkyl” refers to an alkyl substituent which is further substituted with one or more amino groups. The term “hydroxyalkyl” or “hydroxylalkyl” refers to an alkyl substituent which is further substituted with one or more hydroxyl groups. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

Alkylating agents are any reagent that is capable of effecting the alkylation of the functional group at issue (e.g., oxygen atom of an alcohol, nitrogen atom of an amino group). Alkylating agents are known in the art, including in the references cited herein, and include alkyl halides (e.g., methyl iodide, benzyl bromide or chloride), alkyl sulfates (e.g., methyl sulfate), or other alkyl group-leaving group combinations known in the art. Leaving groups are any stable species that can detach from a molecule during a reaction (e.g., elimination reaction, substitution reaction) and are known in the art, including in the references cited herein, and include halides (e.g., I—, Cl—, Br—, F—), hydroxy, alkoxy (e.g., —OMe, —O-t-Bu), acyloxy anions (e.g., —OAc, —OC(O)CF₃), sulfonates (e.g., mesyl, tosyl), acetamides (e.g., —NHC(O)Me), carbamates (e.g., N(Me)C(O)Ot-Bu), phosphonates (e.g., —OP(O)(OEt)₂), water or alcohols (protic conditions), and the like.

In certain embodiments, substituents on any group (such as, for example, alkyl, alkenyl, alkynyl, aryl, aralkyl, heteroaryl, heteroaralkyl, cycloalkyl, heterocycloalkyl) can be at any atom of that group, wherein any group that can be substituted (such as, for example, alkyl, alkenyl, alkynyl, aryl, aralkyl, heteroaryl, heteroaralkyl, cycloalkyl, heterocycloalkyl) can be optionally substituted with one or more substituents (which may be the same or different), each replacing a hydrogen atom. Examples of suitable substituents include, but are not limited to alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carbamido, carbamyl, thioureido, thiocyanato, sulfoamido, sulfonylalkyl, sulfonylaryl, or mercaptoalkoxy.

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. The terms “optionally substituted”, “optionally substituted alkyl,” “optionally substituted “optionally substituted alkenyl,” “optionally substituted alkynyl”, “optionally substituted cycloalkyl,” “optionally substituted cycloalkenyl,” “optionally substituted aryl”, “optionally substituted heteroaryl,” “optionally substituted aralkyl”, “optionally substituted heteroaralkyl,” “optionally substituted heterocycloalkyl,” and any other optionally substituted group as used herein, refer to groups that are substituted or unsubstituted by independent replacement of one, two, or three or more of the hydrogen atoms thereon with substituents including, but not limited to:

—F, —Cl, —Br, —I,

—OH, protected hydroxy,

—NO₂, —CN,

—NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino,

—O—C₁-C₁₂-alkyl, —O—C₂-C₁₂-alkenyl, —O—C₂-C₁₂-alkenyl, —O—C₃-C₁₂-cycloalkyl, —O-aryl, —O-heteroaryl, —O-heterocycloalkyl,

—C(O)—C₁-C₁₂-alkyl, —C(O)—C₂-C₁₂-alkenyl, —C(O)—C₂-C₁₂-alkenyl, —C(O)—C₃-C₁₂-cycloalkyl, —C(O)-aryl, —C(O)-heteroaryl, —C(O)-heterocycloalkyl,

—CONH₂, —CONH—C₁-C₁₂-alkyl, —CONH—C₂-C₁₂-alkenyl, —CONH—C₂-C₁₂-alkenyl, —CONH—C₃-C₁₂-cycloalkyl, —CONH-aryl, —CONH-heteroaryl, —CONH-heterocycloalkyl,

—OCO₂—C₁-C₁₂-alkyl, —OCO₂—C₂-C₁₂-alkenyl, —OCO₂—C₂-C₁₂-alkenyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkenyl, —OCONH—C₃-C₁₂-cycloalkyl, —OCONH— aryl, —OCONH— heteroaryl, —OCONH— heterocycloalkyl,

—NHC(O)—C₁-C₁₂-alkyl, —NHC(O)—C₂-C₁₂-alkenyl, —NHC(O)—C₂-C₁₂-alkenyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkenyl, —NHCO₂—C₃-C₁₂-cycloalkyl, —NHCO₂-aryl, —NHCO₂— heteroaryl, —NHCO₂— heterocycloalkyl, —NHC(O)NH₂, —NHC(O)NH—C₁-C₁₂-alkyl, —NHC(O)NH—C₂-C₁₂-alkenyl, —NHC(O)NH—C₂-C₁₂-alkenyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₁₂-alkenyl, —NHC(S)NH—C₂-C₁₂-alkenyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₁₂-alkenyl, —NHC(NH)NH—C₂-C₁₂-alkenyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₁₂-alkenyl, —NHC(NH)—C₂-C₁₂-alkenyl, —NHC(NH)—C₃-C₁₂-cycloalkyl, —NHC(NH)-aryl, —NHC(NH)-heteroaryl, —NHC(NH)-heterocycloalkyl,

—C(NH)NH—C₁-C₁₂-alkyl, —C(NH)NH—C₂-C₁₂-alkenyl, —C(NH)NH—C₂-C₁₂-alkenyl, —C(NH)NH—C₃-C₁₂-cycloalkyl, —C(NH)NH-aryl, —C(NH)NH-heteroaryl, —C(NH)NH-heterocycloalkyl,

—S(O)—C₁-C₁₂-alkyl, —S(O)—C₂-C₁₂-alkenyl, —S(O)—C₂-C₁₂-alkenyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₁₂-alkenyl, —SO₂NH—C₂-C₁₂-alkenyl, —SO₂NH—C₃-C₁₂-cycloalkyl, —SO₂NH-aryl, —SO₂NH— heteroaryl, —SO₂NH— heterocycloalkyl,

—NHSO₂—C₁-C₁₂-alkyl, —NHSO₂—C₂-C₁₂-alkenyl, —NHSO₂—C₂-C₁₂-alkenyl, —NHSO₂—C₃-C₁₂-cycloalkyl, —NHSO₂-aryl, —NHSO₂-heteroaryl, —NHSO₂-heterocycloalkyl,

—CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkenyl, —S—C₃-C₁₂-cycloalkyl, —S-aryl, —S-heteroaryl, —S-heterocycloalkyl, or methylthiomethyl.

Compounds of the invention can be made by means known in the art of organic synthesis. Methods for optimizing reaction conditions, if necessary minimizing competing by products, are known in the art. Reaction optimization and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jähnisch, K et al, Angew. Chem. Int. Ed. Engl. 2004 43: 406; and references therein). Additional reaction schemes and protocols may be determined by the skilled artesian by use of commercially available structure-searchable database software, for instance, SciFinder® (CAS division of the American Chemical Society) and CrossFire Beilstein® (Elsevier MDL), or by appropriate keyword searching using an interne search engine such as Google® or keyword databases such as the US Patent and Trademark Office text database.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3.sup.rd edition, John Wiley and Sons, Inc., 1999. The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired bridged macrocyclic products of the present invention. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this invention may be modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

The compounds of the invention are defined herein by their chemical structures and/or chemical names. Where a compound is referred to by both a chemical structure and a chemical name, and the chemical structure and chemical name conflict, the chemical structure is determinative of the compound's identity.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The compounds herein may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present invention. The compounds herein may also be represented in multiple tautomeric forms, in such instances, the invention expressly includes all tautomeric forms of the compounds described herein, even though only a single tautomeric form may be represented. All such isomeric forms of such compounds herein are expressly included in the present invention. All crystal forms and polymorphs of the compounds described herein are expressly included in the present invention. Also embodied are extracts and fractions comprising compounds of the invention. The term isomers is intended to include diastereoisomers, enantiomers, regioisomers, structural isomers, rotational isomers, tautomers, and the like. For compounds which contain one or more stereogenic centers, e.g., chiral compounds, the methods of the invention may be carried out with an enantiomerically enriched compound, a racemate, or a mixture of diastereomers.

Preferred enantiomerically enriched compounds have an enantiomeric excess of 50% or more, more preferably the compound has an enantiomeric excess of 60%, 70%, 80%, 90%, 95%, 98%, or 99% or more. In preferred embodiments, only one enantiomer or diastereomer of a chiral compound of the invention is administered to cells or a subject.

Another object of the present invention is the use of a compound as described herein (e.g., of any formulae herein) in the manufacture of a medicament for use in the treatment of a cell proliferation disorder or disease. Another object of the present invention is the use of a compound as described herein (e.g., of any formulae herein) for use in the treatment of a cell proliferation disorder or disease.

### Methods of Assaying Neoplastic Cell Growth or Proliferation

As reported herein, inhibition of LDHA was found to reduce the growth and proliferation of neoplastic cells. Accordingly, the invention provides for the identification and use of therapeutic compounds (e.g., compounds of Formula I-IV, FX11, or derivatives thereof) that inhibit LDHA activity for the treatment of neoplasia. Compounds that inhibit LDHA are known in the art and are described, for example, by Deck et al., J. Med. Chem., 1998, 41 (20), pp 3879-3887. Additional compounds, including derivatives of FX11, may be identified in an assay for LDH or LDHA activity. Assays for LDHA activity are known in the art and described, for example, by Deck (supra). Assays for LDH activity are also known in the art and described, for example by Babson, A L and Babson, S R. (1973) Kinetic Colorimetric Measurement of Serum

Lactate Dehydrogenase Activity. Clin Chem. 19(7):766-9; Karlsen R L, Norgaard L, Guldbrandsen E B (1981). A rapid method for the determination of urea stable lactate dehydrogenase on the ‘Cobas Bio’ centrifugal analyser. Scand J Clin Lab Invest. 41(5):513-6; Coley H M, Lewandowicz G, Sargent J M, Verrill M W (1997). Chemosensitivity testing of fresh and continuous tumor cell cultures using lactate dehydrogenase. Anticancer Res. 17(1A):231-6; and Howell et al., Clinical Chemistry 25: 269-272, 1979. Kits for measuring LDH activity are also commercially available, for example, the QuantiChrom™ Lactate Dehydrogenase Kit by BioAssay Systems.

If desired, compounds that inhibit LDHA are tested for efficacy in inhibiting neoplastic cell growth in vitro and/or in vivo. In various embodiments, such inhibitors are assayed for activity under normoxic or hypoxic conditions, and/or in the presence or absence of glucose, or another energy source. In one approach, a candidate compound is added to the culture media of a neoplastic cell. Cell survival is then evaluated in the presence or the absence of the compound under normoxic and/or hypoxic conditions, and/or in the presence or absence of glucose. A compound that reduces the survival of a cell, particularly under hypoxic conditions, is identified as useful in the methods of the invention. Compounds that selectively reduce the survival of a cell under hypoxic conditions without substantially effecting the survival of a cell under normoxic conditions are particularly useful. Neoplastic cells suitable for such screens include, but are not limited to, human glioblastoma U-87-MG cells, human pancreatic cell lines E3LZ10.7 and P10, Ramos Burkitt lymphoma cells, human P198 pancreatic cancer cells, and human P493 B cells are available through the ATCC. The selectivity of such compounds suggests that they are unlikely to adversely effect normal cells; thus, such compounds are unlikely to cause the adverse side-effects typically associated with conventional chemotherapeutics. Therapeutics useful in the methods of the invention include, but are not limited to, those that alter a LDHA biological activity associated with cell proliferation, glycolytic metabolism, or those that have an anti-neoplastic activity.

Selected compounds desirably reduce the survival, growth, or proliferation of neoplastic cells. Methods of assaying cell growth and proliferation are known in the art and are described herein. (See, for example, Kittler et al. (Nature. 432 (7020):1036-40, 2004) and by Miyamoto et al. (Nature 416(6883):865-9, 2002)). Assays for cell proliferation generally involve the measurement of DNA synthesis during cell replication. In one embodiment, DNA synthesis is detected using labeled DNA precursors, such as ([³H]-thymidine or 5-bromo-2′-deoxyuridine [BrdU], which are added to cells (or animals) and then the incorporation of these precursors into genomic DNA during the S phase of the cell cycle (replication) is detected (Ruefli-Brasse et al., Science 302(5650):1581-4, 2003; Gu et al., Science 302 (5644):445-9, 2003).

Candidate compounds that reduce the survival of a neoplastic cell in the presence or absence of glucose, or under normoxic or hypoxic conditions are particularly useful as anti-neoplasm therapeutics. Assays for measuring cell viability are known in the art, and are described, for example, by Crouch et al. (J. Immunol. Meth. 160, 81-8); Kangas et al. (Med. Biol. 62, 338-43, 1984); Lundin et al., (Meth. Enzymol. 133, 27-42, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull et al., Heterocyclic Chem. 25, 911, 1988). Assays for cell viability are also available commercially. These assays include CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehydrogenase (LDH) cytotoxicity assay.

Candidate compounds that increase neoplastic cell death, particularly under hypoxic conditions, (e.g., increase apoptosis), or in the presence or absence of glucose are also useful as anti-neoplasm therapeutics. Assays for measuring cell apoptosis are known to the skilled artisan. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

### Screening Assays

Compositions of the invention are useful for the high-throughput low-cost screening of candidate compounds that are useful for reducing the survival of a neoplastic cell. Such compounds include those that inhibit an enzyme that functions in metabolism (e.g., lactate dehydrogenase A, nicotinamide phosphoribosyltransferase). Compounds that reduce glycolysis (e.g., FX11, E, or a derivative thereof) may be tested alone or in combination with other compounds that modulate metabolism. Compounds that inhibit LDHA activity, nicotinamide phosphoribosyltransferase activity, or the activity of another enzyme involved in glycolysis or glutaminolysis are identified as useful in the methods of the invention. Any number of methods are available for carrying out screening assays to identify new candidate compounds. In one embodiment, a compound that increases cell death of a neoplastic cell characterized by glycolytic metabolism is considered useful in the invention; such a candidate compound may be used, for example, as a therapeutic to prevent, delay, ameliorate, stabilize, or treat a neoplasia. Such therapeutic compounds are useful in vivo.

In one example, candidate compounds are screened for those that specifically bind to and inhibit a LDHA polypeptide or fragment thereof. Such an interaction can be readily assayed using any number of standard binding techniques and functional assays (e.g., those described in Ausubel et al., supra). In one embodiment, a compound that binds LDHA is assayed in a neoplastic cell in vitro for the ability to inhibit LDHA activity and reduce neoplastic cell survival. In another example, a candidate compound that binds to LDHA is identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for LDHA is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds and chimeric polypeptides identified using such methods are then assayed for their effect on cell survival as described herein.

In yet another example, the compound, e.g., the substrate, is coupled to a radioisotope or enzymatic label such that binding of the compound to the substrate, (e.g., the LDHA) can be determined by detecting the labeled compound, e.g., substrate, in a complex. For example, compounds can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

In yet another embodiment, a cell-free assay is provided in which an LDHA polypeptide or a biologically active portion thereof is contacted with a test compound and the ability of the test compound to bind to the polypeptide thereof is evaluated.

The interaction between two molecules can also be detected, e.g., using fluorescence energy transfer (FET) (see, for example, Lakowicz et al., U.S. Pat. No. 5,631,169; Stavrianopoulos et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of a test compound to bind to an LDHA polypeptide can be accomplished using real-time Biomolecular Interaction Analysis (BIA) (see, e.g., Sjolander, S, and Urbaniczky, C., Anal. Chem. 63:2338-2345, 1991; and Szabo et al., Curr. Opin. Struct. Biol. 5:699-705, 1995). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

It may be desirable to immobilize either the candidate compound or LDHA to facilitate separation of complexed from uncomplexed forms of one or both of the proteins, as well as to accommodate automation of the assay. Other techniques for immobilizing a complex of a test compound and an LDHA polypeptide on matrices include using conjugation of biotin and streptavidin. For example, biotinylated proteins can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

Compounds isolated by this method (or any other appropriate method) may, if desired, be further purified (e.g., by high performance liquid chromatography). In addition, these candidate compounds may be tested for their ability to inhibit the activity of a LDHA polypeptide (e.g., as described herein). Compounds that bind and inhibit LDHA isolated by this approach may also be used, for example, as therapeutics to treat neoplasia in a subject. Compounds that are identified as binding to a polypeptide of the invention with an affinity constant less than or equal to 10 mM are considered particularly useful in the invention. Alternatively, any in vivo protein interaction detection system, for example, any two-hybrid assay may be utilized.

In another embodiment, a LDHA nucleic acid described herein is expressed as a transcriptional or translational fusion with a detectable reporter, and expressed in an isolated cell (e.g., mammalian or insect cell) under the control of an endogenous or a heterologous promoter. The cell expressing the fusion protein is then contacted with a candidate compound, and the expression of the detectable reporter in that cell is compared to the expression of the detectable reporter in an untreated control cell. A candidate compound that decreases the expression of the LDHA detectable reporter is a compound that is useful for the treatment of a neoplasia.

One skilled in the art appreciates that the effects of a candidate compound on LDHA expression or biological activity are typically compared to the expression or activity of LDHA in the absence of the candidate compound. Thus, the screening methods include comparing the value of a cell modulated by a candidate compound to a reference value of an untreated control cell.

Expression levels can be compared by procedures well known in the art such as RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, and ELISA, microarray analysis, or colorimetric assays, such as the Bradford Assay and Lowry Assay. Changes in neoplastic cell growth further comprise values and/or profiles that can be assayed by methods of the invention by any method known in the art, including x-ray, sonogram, ultrasound, MRI, or PET scan.

Molecules that alter LDHA expression or activity include organic molecules, peptides, peptide mimetics, polypeptides, nucleic acids, and antibodies that bind to an LDHA nucleic acid sequence or polypeptide and alter its expression or biological activity are preferred.

Each of the DNA sequences listed herein may also be used in the discovery and development of a therapeutic compound for the treatment of a neoplasia. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

Small molecules of the invention preferably have a molecular weight below 2,000 daltons, more preferably between 300 and 1,000 daltons, and most preferably between 400 and 700 daltons. It is preferred that these small molecules are organic molecules.

### Test Compounds and Extracts

In general, compounds capable of altering the activity of an LDHA polypeptide are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to increase the activity of an LDHA polypeptide, or binding to an LDHA polypeptide, further fractionation of the positive lead extract is necessary to isolate chemical constituents responsible for the observed effect. Thus, the goal of the extraction, fractionation, and purification process is the careful characterization and identification of a chemical entity within the crude extract that alter the activity of an LDHA polypeptide. Methods of fractionation and purification of such heterogenous extracts are known in the art. If desired, compounds shown to be useful as therapeutics for the treatment of a neoplasia are chemically modified according to methods known in the art.

If desired, candidate compounds selected using any of the screening methods described herein are tested for their efficacy using animal models of neoplasia. In one approach, the effect of a candidate compound on tumor load is analyzed in mice injected with human neoplastic cells. The neoplastic cell is allowed to grow to form a mass, preferably a mass characterized as PET positive and/or as having a glycolytic metabolism. The mice are then treated with a candidate compound or vehicle (PBS) daily for a period of time to be empirically determined. Mice are euthanized and the neoplastic tissue is collected. The mass of the neoplastic tissue in mice treated with the selected candidate compounds is compared to the mass of neoplastic tissue present in corresponding control mice.

In another approach, mice are injected with neoplastic human cells. The mice containing the neoplastic cells are then injected (e.g., intraperitoneally) with vehicle (PBS) or candidate compound daily for a period of time to be empirically determined. Mice are then euthanized and the neoplastic tissues are collected and analyzed for LDHA nucleic acid or protein levels using methods described herein. Compounds that decrease LDHA mRNA or protein expression relative to control levels are expected to be efficacious for the treatment of a neoplasm in a subject (e.g., a human patient).

Preferably, compounds selected according to the methods of the invention reduce the growth, proliferation, or severity of the neoplasm by at least 10%, 25%, or 50%, or by as much as 75%, 85%, or 95% when compared to a control.

### Inhibitory Nucleic Acid Molecules

Inhibitory nucleic acid molecules (e.g., siRNAs, shRNAs, antisense) are useful for reducing the expression of a LDHA polypeptide. Accordingly, the invention provides inhibitory nucleic acid molecules that are useful for decreasing the expression of a polypeptide of interest (e.g., LDHA). Inhibitory nucleic acid molecules include, but are not limited to double-stranded RNAs, antisense RNAs, and siRNAs, or portions thereof. As reported in more detail below, the inhibition of LDHA expression by an siRNA reduced the survival of neoplastic cells.

The inhibitory nucleic acids of the present invention may be employed in double-stranded RNAs for RNA interference (RNAi)-mediated knock-down of LDHA expression. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Harmon, Nature 418:244-251, 2002). RNA interference (RNAi) provides for the targeting of specific mRNAs for degradation by complementary short-interfering RNAs (siRNAs). RNAi is a useful therapeutic approach for gene silencing. The general mechanism of RNAi involves the cleavage of double-stranded RNA (dsRNA) to short 21-23-nt siRNAs. This processing event is catalyzed by Dicer, a highly conserved, dsRNA-specific endonuclease that is a member of the RNase III family. Processing by Dicer results in siRNA duplexes that have 5′-phosphate and 3′-hydroxyl termini, and subsequently, these siRNAs are recognized by the RNA-induced silencing complex (RISC). Active RISC complexes (RISC*) promote the unwinding of the siRNA through an ATP-dependent process, and the unwound antisense strand guides RISC to the complementary mRNA. The targeted mRNA is then cleaved by RISC at a single site that is defined with regard to where the 5′-end of the antisense strand is bound to the mRNA target sequence. siRNAs use as therapeutic agents is improved by modifications that enhance the stability of siRNAs.

In one embodiment of the invention, a double-stranded RNA (dsRNA) molecule includes between eight and twenty-five consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Given the sequence of a mammalian gene (e.g., LDHA), siRNAs may be designed to inactivate that gene. For example, for a gene that consists of 2000 nucleotides, approximately 1,978 different twenty-two nucleotide oligomers could be designed; this assumes that each oligomer has a two base pair 3′ overhang, and that each siRNA is one nucleotide residue from the neighboring siRNA. To effectively silence the gene, only a few of these twenty-two nucleotide oligomers would be needed; approximately 1, 5, 10, or 12 siRNAs could be sufficient to significantly reduce mammalian gene activity. In one embodiment, an siRNA that targets LDHA is transferred into a mammalian cell in culture, and the effect of the siRNAs on the LDHA expression or activity in the cultured cells is assayed. Methods for assaying LDHA activity are known in the art and are described herein. Methods for assaying LDHA activity are described, for example, by Aicher et al. (J. Med. Chem. 43:236-249, 2000). Alternatively, siRNAs could be injected into an animal, for example, into the blood stream (McCaffrey et al., Nature 418:38-92002).

Unmodified siRNAs may be limited in their therapeutic applications by their sensitivity towards nucleases. Chemical strategies to improve stability such as the modification of the deoxyribo/ribo sugar and the heterocyclic base are known in the art, as are the modification or replacement of the internucleotide phosphodiester linkage. Methods for enhancing siRNA stability are described, for example, by Chiu et al., (RNA 9:1034-1048, 2003); Layzer, et al. (RNA 10, 766-771, 2004); and by Morrissey et al., (Nature Biotechnology 23, 1002-1007, 2005). In various approaches, fully modified 2′-O-propyl and 2′-O-pentyl oligoribonucleotides are used to enhance inhibitory nucleic acid stability chemical modifications that stabilized interactions between A-U base pairs; thioate linkages (P-S) are integrated into the backbone; uridine and cytidine in the antisense strand of siRNA are replaced with 2′-fluoro-uridine (2′-FU) and 2′-fluoro-cytidine (2′-FC), respectively, which have a fluoro group at the 2′-position in place of the 2′-OH; 5-bromo-uridine (U[5Br]), 5-iodo-uridine (U[5I]), or 2,6-diaminopurine (DAP) are included in the siRNA. Such approaches are useful for enhancing siRNA stability. Other useful modifications for enhancing siRNA stability are described below.

In another approach, antisense oligonucleotides are used to decrease the expression of LDHA. The efficacy of antisense technology lies in the specific binding of an oligoribonucleotide to its target sequence. The formation of a duplex between an antisense oligomer and its target sequence prevents gene expression by interfering with subsequent processing, transport or translation, or by degradation of the RNA via RNase H. The therapeutic efficacy of antisense molecules is improved by modifications that enhance the stability of the antisense molecule.

### Modifications to Enhance Inhibitory Nucleic Acid Molecule Stability

As is known in the art, a nucleoside is a nucleobase-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; open linear structures are generally preferred. Within the oligonucleotide structure, the phosphate groups are commonly referred to as forming the backbone of the oligonucleotide. The normal linkage or backbone of RNA and DNA is a 3′ to 5′ phosphodiester linkage.

Specific examples of preferred inhibitory nucleic acid molecules useful in this invention include oligonucleotides containing modified backbones or non-natural internucleoside linkages. As defined in this specification, inhibitory nucleic acid molecules having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be inhibitory nucleic acid molecules.

Inhibitory nucleic acid molecules that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriest-ers, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Inhibitory nucleic acid molecules having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other inhibitory nucleic acid molecules, both the sugar and the internucleoside linkage, i.e., the backbone, are replaced with novel groups. One such inhibitory nucleic acid molecules, is referred to as a Peptide Nucleic Acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Methods for making and using these nucleobase oligomers are described, for example, in “Peptide Nucleic Acids: Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In particular embodiments of the invention, the nucleobase oligomers have phosphorothioate backbones and nucleosides with heteroatom backbones, and in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂—, and —O—N(CH₃)—CH₂—CH₂—. In other embodiments, the oligonucleotides have morpholino backbone structures described in U.S. Pat. No. 5,034,506.

Inhibitory nucleic acid molecules may also contain one or more substituted sugar moieties. Inhibitory nucleic acid molecules comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁ to C₁₀ alkyl or C₂ to C₁₀ alkenyl and alkynyl. Particularly preferred are O[(CH₂)ₙO]ₙCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred nucleobase oligomers include one of the following at the 2′ position: C₁ to C₁₀ lower alkyl, substituted lower alkyl, alkaryl, aralkyl, O-alkaryl, or O-aralkyl, SH, SCH₃, OCN, Cl, Br, CN, CF₃, OCF₃, SOCH₃, SO₂CH₃, ONO₂, NO₂, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleobase oligomer, or a group for improving the pharmacodynamic properties of an nucleobase oligomer, and other substituents having similar properties. Preferred modifications are 2′-O-methyl and 2′-methoxyethoxy (2′-O—CH₂CH₂OCH₃, also known as 2′-O-(2-methoxyethyl) or 2′-MOE). Another desirable modification is 2′-dimethylaminooxyethoxy (i.e., O(CH₂)₂ON(CH₃)₂), also known as 2′-DMAOE. Other modifications include, 2′-aminopropoxy (2′-OCH₂CH.₂CH₂NH₂) and 2′-fluoro (2′-F). Similar modifications may also be made at other positions on an oligonucleotide or other nucleobase oligomer, particularly the 3′ position of the sugar on the 3′ terminal nucleotide or in 2′-5′ linked oligonucleotides and the 5′ position of 5′ terminal nucleotide. Inhibitory nucleic acid molecules may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Inhibitory nucleic acid molecules may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil (pseudouracil); 4-thiouracil; 8-halo, 8-amino, 8-thiol, 8-thioalkyl, 8-hydroxyl and other 8-substituted adenines and guanines; 5-halo (e.g., 5-bromo), 5-trifluoromethyl and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of an antisense oligonucleotide of the invention. These include 5-substituted pyrimidines, 6-azapyrimidines, and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are desirable base substitutions, even more particularly when combined with 2′-O-methoxyethyl or 2′-O-methyl sugar modifications. Representative United States patents that teach the preparation of certain of the above noted modified nucleobases as well as other modified nucleobases include U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692, each of which is herein incorporated by reference.

Another modification of an inhibitory nucleic acid of the invention involves chemically linking to the nucleobase oligomer one or more moieties or conjugates that enhance the activity, cellular distribution, or cellular uptake of the oligonucleotide. Such moieties include but are not limited to lipid moieties such as a cholesterol moiety (Letsinger et al., Proc. Natl. Acad. Sci. USA, 86:6553-6556, 1989), cholic acid (Manoharan et al., Bioorg. Med. Chem. Let, 4:1053-1060, 1994), a thioether, e.g., hexyl-5-tritylthiol (Manoharan et al., Ann. N.Y. Acad. Sci., 660:306-309, 1992; Manoharan et al., Bioorg. Med. Chem. Let., 3:2765-2770, 1993), a thiocholesterol (Oberhauser et al., Nucl. Acids Res., 20:533-538: 1992), an aliphatic chain, e.g., dodecandiol or undecyl residues (Saison-Behmoaras et al., EMBO J., 10:1111-1118, 1991; Kabanov et al., FEBS Lett., 259:327-330, 1990; Svinarchuk et al., Biochimie, 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett., 36:3651-3654, 1995; Shea et al., Nucl. Acids Res., 18:3777-3783, 1990), a polyamine or a polyethylene glycol chain (Manoharan et al., Nucleosides & Nucleotides, 14:969-973, 1995), or adamantane acetic acid (Manoharan et al., Tetrahedron Lett., 36:3651-3654, 1995), a palmityl moiety (Mishra et al., Biochim. Biophys. Acta, 1264:229-237, 1995), or an octadecylamine or hexylamino-carbonyl-oxycholesterol moiety (Crooke et al., J. Pharmacol. Exp. Ther., 277:923-937, 1996. Representative United States patents that teach the preparation of such nucleobase oligomer conjugates include U.S. Pat. Nos. 4,587,044; 4,605,735; 4,667,025; 4,762,779; 4,789,737; 4,824,941; 4,828,979; 4,835,263; 4,876,335; 4,904,582; 4,948,882; 4,958,013; 5,082,830; 5,109,124; 5,112,963; 5,118,802; 5,138,045; 5,214,136; 5,218,105; 5,245,022; 5,254,469; 5,258,506; 5,262,536; 5,272,250; 5,292,873; 5,317,098; 5,371,241, 5,391,723; 5,414,077; 5,416,203, 5,451,463; 5,486,603; 5,510,475; 5,512,439; 5,512,667; 5,514,785; 5,525,465; 5,541,313; 5,545,730; 5,552,538; 5,565,552; 5,567,810; 5,574,142; 5,578,717; 5,578,718; 5,580,731; 5,585,481; 5,587,371; 5,591,584; 5,595,726; 5,597,696; 5,599,923; 5,599,928; 5,608,046; and 5,688,941, each of which is herein incorporated by reference.

The present invention also includes inhibitory nucleic acid molecules that are chimeric compounds. “Chimeric” inhibitory nucleic acid molecules are inhibitory nucleic acid molecules, particularly oligonucleotides, that contain two or more chemically distinct regions, each made up of at least one monomer unit, i.e., a nucleotide in the case of an oligonucleotide. These ₂ typically contain at least one region where the nucleobase oligomer is modified to confer, upon the ₂, increased resistance to nuclease degradation, increased cellular uptake, and/or increased binding affinity for the target nucleic acid. An additional region of the inhibitory nucleic acid molecule, such as an antisense molecule, may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of nucleobase oligomer inhibition of gene expression. Consequently, comparable results can often be obtained with shorter inhibitory nucleic acid molecules when chimeric inhibitory nucleic acid molecules are used, compared to phosphorothioate deoxyoligonucleotides hybridizing to the same target region.

Chimeric inhibitory nucleic acid molecules of the invention may be formed as composite structures of two or more nucleobase oligomers as described above. Such nucleobase oligomers, when oligonucleotides, have also been referred to in the art as hybrids or gapmers. Representative United States patents that teach the preparation of such hybrid structures include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The inhibitory nucleic acid molecules used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The inhibitory nucleic acid molecules of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The inhibitory nucleic acid molecules of the invention encompass any pharmaceutically acceptable salts, esters, or salts of such esters, or any other compound that, upon administration to an animal, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

### LDHA Antibodies

Antibodies are well known to those of ordinary skill in the science of immunology. Particularly useful in the methods of the invention are antibodies that specifically bind a LDHA polypeptide and inhibit the activity of the polypeptide. Antibodies that inhibit the activity of LDHA are useful for the treatment of a neoplasia. Accordingly, an antibody that specifically binds LDHA is assayed for such activity as described herein.

As used herein, the term “antibody” means not only intact antibody molecules, but also fragments of antibody molecules that retain immunogen binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments which lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies.

Unconventional antibodies include, but are not limited to, nanobodies, linear antibodies (Zapata et al., Protein Eng. 8(10): 1057-1062, 1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody since small molecules of ˜60-100 kDa in size provide faster blood clearance and rapid tissue uptake See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making and using unconventional antibodies have been described. Bispecific antibodies produced using leucine zippers are described by Kostelny et al. (J. Immunol. 148(5):1547-1553, 1992). Diabody technology is described by Hollinger et al. (Proc. Natl. Acad. Sci. USA 90:6444-6448, 1993). Another strategy for making bispecific antibody fragments by the use of single-chain Fv (sFv) diners is described by Gruber et al. (J. Immunol. 152:5368, 1994). Trispecific antibodies are described by Tutt et al. (J. Immunol. 147:60, 1991). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

In one embodiment, an antibody that binds an LDHA polypeptide is monoclonal. Alternatively, the anti-LDHA antibody is a polyclonal antibody. The preparation and use of polyclonal antibodies are also known the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fe” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab′ fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing LDHA polypeptides, or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding an LDHA polypeptide, or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding an LDHA polypeptide, or immunogenic fragments thereof, can be expressed in cells in vitro, followed by isolation of the receptor and administration of the receptor to a suitable host in which antibodies are raised.

Using either approach, antibodies can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition; e.g., Pristane.

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

### Pharmaceutical Therapeutics

The invention provides a simple means for identifying compositions (including nucleic acids, peptides, small molecule inhibitors, and mimetics) capable of acting as therapeutics for the treatment of a neoplasia. Using the methods of the invention, FX11, which selectively inhibits lactate dehydrogenase A, was identified as a compound that inhibits glycolysis, which is the preferred metabolic pathway in neoplastic cells. Using the methods described herein, other compounds (e.g., compounds of Formulas I-IV) having the ability to inhibit LDHA and reduce the survival of a neoplastic cell may be identified. A compound discovered to have medicinal value using the methods described herein is useful as a drug or as information for structural modification of existing compounds, e.g., by rational drug design. Such methods are useful for screening compounds having an effect on the expression or activity of an LDHA polypeptide.

For therapeutic uses, the compositions or agents identified using the methods disclosed herein may be administered systemically, for example, formulated in a pharmaceutically-acceptable buffer such as physiological saline. For the treatment of cancer, the compounds of the invention are preferably delivered systemically by intravenous injection. Other routes of administration include, for example, subcutaneous, intravenous, interperitoneally, intramuscular, or intradermal injections that provide continuous, sustained levels of the drug in the patient. In another embodiment, a compound of the invention is administered locally via catheter. Treatment of human patients or other animals will be carried out using a therapeutically effective amount of an anti-neoplasia therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia, although in certain instances lower amounts will be needed because of the increased specificity of the compound. A compound is administered at a dosage that controls the clinical or physiological symptoms of neoplasia as determined by a diagnostic method known to one skilled in the art, or using any that assay that measures the expression or the biological activity of an LDHA polypeptide.

In one embodiment, the present invention provides methods of treating disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a neoplastic disease, disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which LDHA may be implicated.

### Formulation of Pharmaceutical Compositions

The administration of a compound for the treatment of neoplasia may be by any suitable means that results in a concentration of the therapeutic that, combined with other components, is effective in ameliorating, reducing, or stabilizing a neoplasia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target a neoplasia by using carriers or chemical derivatives to deliver the therapeutic agent to a particular cell type (e.g., neoplastic cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the neoplasia therapeutic (s), the composition may include suitable parenterally acceptable carriers and/or excipients. The active anti-neoplasia therapeutic (s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active anti-neoplasia therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms for Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active active anti-neoplasia therapeutic substance). The coating may be applied on the solid dosage form in a similar manner as that described in Encyclopedia of Pharmaceutical Technology, supra.

At least two active anti-neoplasia therapeutics may be mixed together in the tablet, or may be partitioned. In one example, the first active therapeutic is contained on the inside of the tablet, and a second active therapeutic is on the outside, such that a substantial portion of the second active therapeutic is released prior to the release of the first active therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active anti-neoplasia therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Dosage Determination

Those of skill in the art will recognize that the best treatment regimens for using compounds of the present invention (e.g., inhibitors of a LDHA) to treat a neoplasia can be straightforwardly determined. This is not a question of experimentation, but rather one of optimization, which is routinely conducted in the medical arts. In vivo studies in nude mice often provide a starting point from which to begin to optimize the dosage and delivery regimes. The frequency of injection will initially be once a week, as has been done in some mice studies. However, this frequency might be optimally adjusted from one day to every two weeks to monthly, depending upon the results obtained from the initial clinical trials and the needs of a particular patient.

Human dosage amounts can initially be determined by extrapolating from the amount of compound used in mice, as a skilled artisan recognizes it is routine in the art to modify the dosage for humans compared to animal models. In one embodiment, a compound of the invention (e.g., a compound of Formula I-IV, FX11, or a derivative thereof) is administered at about 2, 3, 5, 10, 25, 50, 100, 120, or 150 mg/kg/day. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. Of course, this dosage amount may be adjusted upward or downward, as is routinely done in such treatment protocols, depending on the results of the initial clinical trials and the needs of a particular patient.

### Patient Monitoring

The disease state or treatment of a patient having a neoplasia can be monitored using the methods and compositions of the invention. In one embodiment, the metabolic profile of a neoplasia is assayed using a PET scan to identify an alteration in the glycolytic metabolism of the neoplasia. In another embodiment, the expression or activity of an LDHA or nicotinamide phosphoribosyltransferase nucleic acid molecule or polypeptide is monitored using any method known in the art. Neoplastic cells that have increased glycolytic metabolism or that have acquired mutations that permit them to metabolize glucose or glutamine are identified as amenable to treatment with FX11 alone or in combination with FK 866, or with any other conventional chemotherapeutic agent. The efficacy of such treatment is then monitored, for example, by assaying for a reduction in glycolytic metabolism, by assaying for a reduction in a signal detected by a PET scan, by assaying for a reduction in tumor size, an increase in tumor cell death, a reduction or stabilization in neoplasia cell proliferation, or by any other method known in the art. In certain neoplasias (e.g., pancreatic cancer, lymphomas following chemotherapy) the presence or persistence of a glycolytic metabolic profile may correlate with adverse outcomes or with resistance to conventional chemotherapeutics, and therefore require more aggressive treatment regiments. In one embodiment, an increase in PET signal or in the expression of LDHA in a patient sample identifies the neoplasia as particularly severe. Therapeutics that decrease PET signal, or that reduce the expression or activity of a LDHA nucleic acid molecule or polypeptide are taken as particularly useful in the invention. Such monitoring may be useful, for example, in assessing the efficacy of a particular drug in a patient or in assessing patient compliance with a treatment regimen.

### Kits

The invention provides kits for the treatment or prevention of a neoplasia or symptoms thereof. In one embodiment, the kit includes an effective amount of an LDHA inhibitor (e.g., compounds of Formula I-IV, FX11, or derivatives thereof) for use in neoplasia. If desired, the LDHA inhibitor is provided alone or in combination with a (NAMPT) inhibitor of NAD⁺ synthesis (e.g., FK 866 or a derivative thereof).

The invention further provides kits for the diagnosis of a neoplasia having a glycolytic metabolism. Such neoplasias are characterized using an imaging reagent of the invention, wherein a compound of Formula I is conjugated at R4 to a detectable moiety. In one embodiment, the compound of Formula I is FX11 and the detectable moiety comprises a radionuclide that is a positron or gamma emitter.

In some embodiments, the kit comprises a sterile container which contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired compositions of the invention are provided together with instructions for administering them to a subject having or at risk of developing a neoplasia. The instructions will generally include information about the use of the compositions for the treatment or prevention of a neoplasia. In other embodiments, the instructions include at least one of the following: description of the composition; dosage schedule and administration for treatment of a neoplasia, or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Diagnostics

Neoplastic tissues that have acquired the ability to metabolize glucose express higher levels of LDHA polypeptides or polynucleotides than corresponding normal tissues. Such neoplastic tissues are detected as positive in a PET scan. Accordingly, a positive PET scan, an increase in levels of expression or activity of an LDHA polypeptide or detection of another marker of glycolytic metabolism are correlated with neoplasia. In one embodiment, detection of a glycolytic metabolic profile identifies the neoplasia as amenable to treatment with a composition of the invention (e.g., FX11, FX11 and FK866). In other embodiments, imaging agents described herein identify particularly aggressive neoplasias, or neoplasias that are resistant to treatment with conventional chemotherapeutics, and thus are useful in diagnosis and treatment selection. Accordingly, the present invention provides a number of diagnostic compositions and methods that are useful for the identification or characterization of a neoplasia.

### Imaging Agents

Certain neoplasias, such as lymphomas, as generally characterized by a predominantly glycolytic metabolism. In other neoplasias, such as pancreatic cancers, detection of a glycolytic metabolism may identify the neoplasia as resistant to conventional chemotherapeutics. In other embodiments, the persistence of a glycolytic metabolism following one, two, or more courses of chemotherapy identifies the neoplasia as particularly aggressive or as resistant to conventional chemotherapy. Accordingly, glycolytic polypeptides, such as LDHA, can serve as specific markers for the diagnosis and/or monitoring of neoplasias. In particular embodiments, the invention provides a compound that binds to an LDHA polypeptide, for example, an FX11 compound that includes a moiety that allows it to be imaged. Preferably, a detectable moiety is conjugated to a carboxyl group present on the FX11 compound. Such detectable moieties are visualized using conventional imaging methods (e.g., PET, Spect-CD, MRI, X-ray). The presence of a glycolytic metabolism in the neoplasia concentrates the compound in the tumor cell, thereby allowing the tumor cells to be visualized.

The ability to image tumor metabolism in vivo has broad application as exemplified by the increasing clinical use of positron emission tomography with [¹⁸F]fluorodeoxyglucose (FDG-PET). The language “effective amount for imaging” of a compound is the amount necessary or sufficient to provide a signal sufficient to visualize the presence or absence of a neoplasm. Neoplasms may be imaged using any method know in the art or described herein, e.g., planar gamma imaging, single photon emission computed tomography (SPECT) and positron emission tomography (PET). The effective amount can vary depending on such factors as the size and weight of the subject, the type of illness, or the particular compound. For example, the choice of the compound can affect what constitutes an “effective amount for imaging”. One of ordinary skill in the art would be able to study the factors contained herein and make the determination regarding the effective amount of the compound without undue experimentation. Imaging can allow for the detection of the presence and/or location of the imaging agent conjugated to a lactate dehydrogenase A inhibitor. Presence can include below the level of detection or not present, and the location can include none. In particular, the invention provides agents, including agents that specifically bind and inhibit a glycolytic enzyme such as LDHA, in an organism and produce a detectable signal that can used to obtain an image of a neoplasm in a subject and determine the presence and location of the neoplasm.

The invention utilizes LDHA binding compounds, including FX11 and derivatives thereof that are easily synthesized and are detectable to an imaging apparatus, e.g., a PET or SPECT instrument.

### Imaging

Generally, imaging techniques involve administering a compound to a subject that can be detected externally to the subject. Images are generated by virtue of differences in the spatial distribution of the imaging agents which accumulate in various locations in a subject. The methods of the present invention, the imaging techniques rely on the compounds being preferentially bound in a subject, e.g., LDHA. The spatial distribution of the imaging agent accumulated in a subject, e.g., tumor volume, may be measured using any suitable means, for example, planar gamma imaging, single photon emission computed tomography (SPECT) and positron emission tomography (PET). Alternatively, imaging techniques that detect fluorescence may be used in the methods of the invention.

The phrase “LDHA binding compound” is understood as a compound that has a sufficient affinity for LDHA such that they are able to be used as imaging agents and/or therapeutic agents. In an embodiment, a LDHA binding compound can be FX11, an analog, or derivative thereof. If desired, such compounds have one or more isotope atoms which may or may not be radioactive (e.g., ³H, ²H, ¹⁴C, ¹³C, ³⁵S, ³²P, ¹²⁵I, and ¹³¹I) introduced into the compound. Such compounds are useful for as diagnostics, in drug metabolism studies, as well as in therapeutic applications. LDHA binding compounds have at least a 10-fold, preferably 100-fold, preferably 1000-fold higher affinity for LDHA as compared to other mammalian lactate dehydrogenases (e.g., LDHB). LDHA binding compounds include, for example, FX11. LDHA binding compounds can be modified to include functional groups to facilitate their use as imaging and/or as therapeutic agents. In preferred embodiments, an imaging moiety is conjugated at the FX11 carboxyl group.

In specific embodiments, FX11 compounds useful for imaging may include a radionuclide (e.g., iodine-¹²³, ¹²⁴ or ¹²⁵). In other embodiments, FX11 is labeled with a radioisotope of fluorine, yttrium, bismuth, or astatine. Among the most commonly used positron-emitting nuclides in PET are ¹¹C, ¹³N, ¹⁵O, and ¹⁸F. Isotopes that decay by electron capture and/or γ emission are used in SPECT, and include, for example, ¹²³I and ¹²⁴I. In still other embodiments, FX11 is labeled with a fluorescent moiety.

The methods of the invention include PET. Specifically, imaging is carried out by scanning the entire patient, or a particular region of the patient using the detection system, and detecting the signal, e.g., the radioisotope signal. The detected signal is then converted into an image. The resultant images should be read by an experienced observer, such as, for example, a physician. The foregoing process is referred to herein as “imaging” the patient. Generally, imaging is carried out about 1 minute to about 48 hours following administration of the compound used in the methods of the invention. The precise timing of the imaging will be dependant upon such factors as the clearance rate of the compound administered, as will be readily apparent to those skilled in the art.

Once an image has been obtained, one of skill in the art will be able to determine the location of the compound. Using this information, the artisan can determine, for example, if a tumor is the extent of the tumor, or the efficacy of treatment which the subject is undergoing. Images obtained at different time points, e.g., 12, 24, 36, 48 or more, hours apart are particularly useful in determining the efficacy of treatment, e.g., therapy and/or chemotherapeutic treatment.

Unlike methods currently used, the imaging methods described herein allow the clinician to distinguish tumors expressing LDHA. In one approach, diagnostic methods of the invention are used to assay the expression of an LDHA polypeptide in a biological sample relative to a reference (e.g., the level of LDHA present in a normal control tissue). In one embodiment, the level of an LDHA polypeptide is detected using an antibody that specifically binds the polypeptide. Such antibodies are useful for the diagnosis of a neoplasia. Methods for measuring an antibody-polypeptide complex include, for example, detection of fluorescence, luminescence, chemiluminescence, absorbance, reflectance, transmittance, birefringence or refractive index. Optical methods include microscopy (both confocal and non-confocal), imaging methods and non-imaging methods. Methods for performing these assays are readily known in the art. Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra. Immunoassays can be used to determine the quantity of LDHA polypeptide in a sample, where an increase in the level of the LDHA polypeptide is diagnostic of a patient having a neoplasia.

In general, the measurement of an LDHA polypeptide or nucleic acid molecule in a subject sample is compared with a diagnostic amount present in a reference. A diagnostic amount distinguishes between a neoplastic tissue and a control tissue. The skilled artisan appreciates that the particular diagnostic amount used can be adjusted to increase sensitivity or specificity of the diagnostic assay depending on the preference of the diagnostician. In general, any significant increase (e.g., at least about 10%, 15%, 30%, 50%, 60%, 75%, 80%, or 90%) in the level of an LDHA polypeptide or nucleic acid molecule in the subject sample relative to a reference may be used to diagnose a neoplasia. In one embodiment, the reference is the level of LDHA polypeptide or nucleic acid molecule present in a control sample obtained from a patient that does not have a neoplasia. In another embodiment, the reference is a baseline level of LDHA polypeptide present in a biologic sample derived from a patient prior to, during, or after treatment for a neoplasia. In yet another embodiment, the reference is a standardized curve.

### Types of Biological Samples

The level of an LDHA polypeptide or nucleic acid molecule can be measured in different types of biologic samples. In one embodiment, the biologic sample is a tissue sample that includes cells of a tissue or organ. Such tissue is obtained, for example, from a biopsy. In another embodiment, the biologic sample is a biologic fluid sample (e.g., blood, blood plasma, serum, urine, seminal fluids, ascites, or cerebrospinal fluid).

### Combination Therapies

Compositions and methods of the invention may be used in combination with any conventional therapy known in the art. Exemplary anti-neoplastic therapies include, for example, chemotherapy, cryotherapy, hormone therapy, radiotherapy, and surgery. A composition of the invention may, if desired, be administered in combination with one or more chemotherapeutics typically used in the treatment of a neoplasm. For example, compositions of the invention (e.g., a compound of Formula I-IV, FX11) may be administered in combination with doxorubicin, bleomycin, vinblastine, and dacarbazine (ABVD). If desired, the combination therapy also includes FK866 or another nicotinamide phosphoribosyltransferase (NAMPT) inhibitor.

Other chemotherapeutics that may be used in a combination of the invention include abiraterone acetate, altretamine, anhydrovinblastine, auristatin, bexarotene, bicalutamide, BMS184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvincaleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, daunorubicin, dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine. Other examples of chemotherapeutic agents can be found in Cancer Principles and Practice of Oncology by V. T. Devita and S. Hellman (editors), 6th edition (Feb. 15, 2001), Lippincott Williams & Wilkins Publishers.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Reduction of LDHA Induced Oxidative Stress and Cell Death

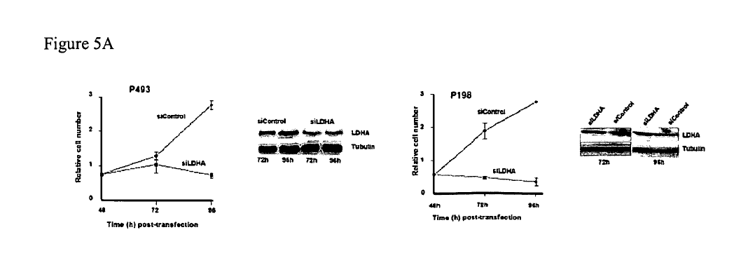

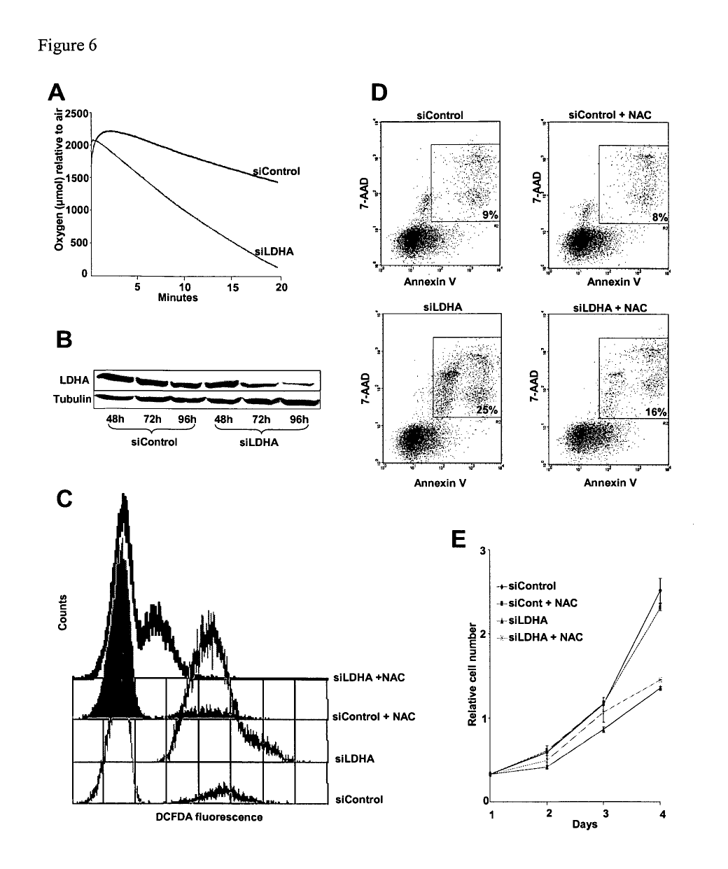

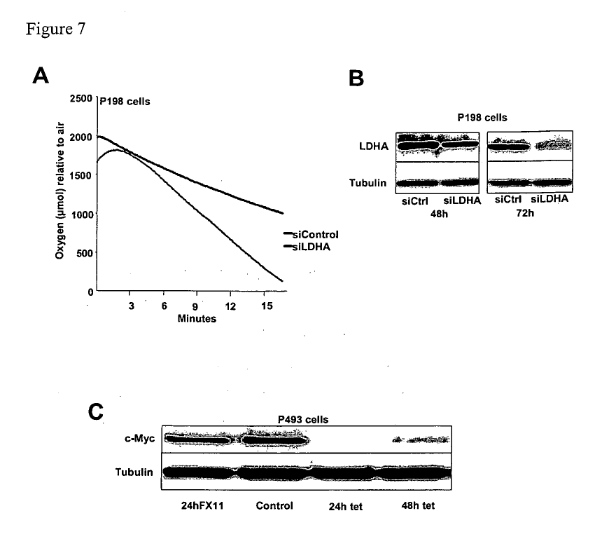

To address the mechanisms of cell death following LDHA reduction by short interfering RNA (siLDHA), the effect of reduced LDHA expression on oxygen consumption by human P493 B lymphoid cells was analysed (FIGS. 5A and 5B). Reduction of LDHA favors the entry of pyruvate into mitochondria for oxidative phosphorylation, thereby enhancing oxygen consumption. Hence, LDHA expression was reduced by siRNA in P493 cells and a corresponding increase in oxygen consumption was observed (FIGS. 6A and 6B). Oxygen consumption was similarly increased in a human pancreatic cancer line treated with siLDHA (FIGS. 7A and 7B).

Enhanced oxygen consumption through reduction of LDHA levels is expected to increase the production of mitochondrial ROS, particularly since glycolysis, which diverts pyruvate to lactate, diminishes cellular oxidative stress (Brand and Hermfisse, 1997). Therefore, the production of ROS by 5-(and-6)-carboxy-2′,7′-dichlorodihydrofluorescein diacetate (DCFDA) fluorescence was determined as measured by flow cytometry (FIG. 6C). Treatment of cells with LDHA siRNA induced significant ROS that could be attenuated by exposing cells to N-acetylcysteine (NAC) (FIG. 6C). Note that while about 60% of siLDHA cells returned to baseline ROS levels, about 40% still displayed ROS, albeit at a reduced level.

Reduction of LDHA expression with siRNA markedly increased cell death that is characterized by enhanced labeling of both 7-AAD and annexin V, which is termed late cell death or necrosis (FIG. 6D). Treatment with the anti-oxidant NAC (20 mM) at 24 hours post-transfection partially reduced cell death, which was also accompanied by a partial rescue of cell proliferation (FIG. 6E).

Reduction of LDHA by siRNA induced oxidative stress and cell death. To determine whether a small molecule inhibitor of LDHA could be used to modulate tumor metabolism, 1200 FDA approved compounds were screened to identify an inhibitor of LDHA. This analysis identified Zaprinast as a weak LDHA inhibitor. As such, a series of compounds generated by Vander Jagt and coworkers, who were specifically interested in targeting malarial LDH (pLDH) (Deck et al., 1998; Yu et al., 2001) was screened. Among the analogs of gossypol, which itself is a toxic inhibitor of LDHA, are dihydroxynapthoates including two compounds 11F (FX11; 2,3-dihydroxy-6-methyl-7-(phenylmethyl)-4-propylnaphthalene-1-carboxylic acid, Pubchem ID: 10498042) and 11e (E; 2,3-dihydroxy-6-methyl-7-(methyl)-4-propylnaphthalene-1-carboxylic acid, Pubchem ID: 10265351). FX11 was selected as a candidate small molecule for inhibiting human LDHA, because it preferentially inhibited LDHA as opposed to LDHB or pLDH (Deck et al., 1998; Yu et al., 2001). Compound E was selected for comparison because it has a lower inhibitory activity versus FX11.

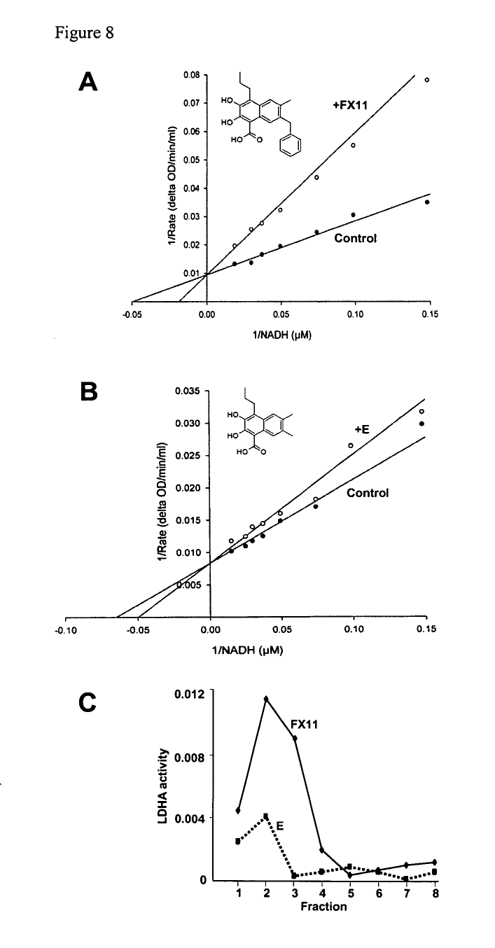

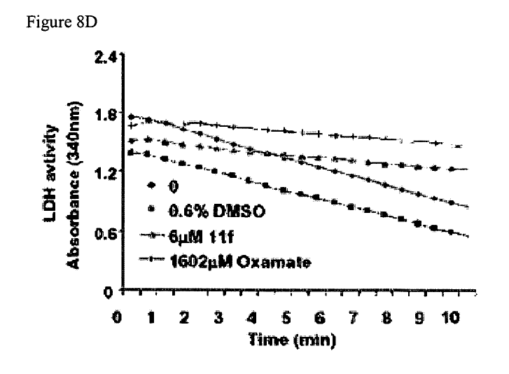

FX11 and E were characterized using purified human liver LDHA. Ki's of 8 μM and >90 μM were found, respectively (FIGS. 8A and 8B). In other studies, pyruvate and NADH were used as the substrates to test the inhibition of LDH by FX11. At the concentration 6 μM, FX11 completely abrogated the LDH activity, the slope of decrease in absorbance at 340 nm (λmax for NADH) is −0.029 in the presence of FX11 as as compared to −0.084 without FX11. The use of oxamate, a pyruvate analogue and inhibitor of LDH as a positive control, required a much higher concentration (1.6 mM) to obtain the same LDH inhibition. FX 11 also inhibited LDH activity in the conversion of lactate to pyruvate but with a higher concentration (34 μM) (FIG. 8D). FX11 is a competitive inhibitor of NADH in the conversion of pyruvate to lactate by LDHA, whereby NADH is converted to NAD⁺. To further document the selective binding of FX11 versus E to LDHA, affinity chromatography was performed with P493 cell lysate using FX11 or E immobilized on Sepharose beads. Equal amounts of cell lysate were loaded onto FX11 or E affinity beads, extensively washed, and the bound LDHA was eluted with 1 mM NADH. The FX11 affinity beads yielded 4-fold more LDHA activity than the beads with immobilized E (FIG. 8C). Collectively, these results indicated that FX11 could bind and inhibit human LDHA enzyme activity. GAPDH is another pivotal glycolytic enzyme that converts NAD⁺ to NADH. The question of whether FX11 could inhibit its NAD⁺-dependent conversion of glyceraldehyde-3-phosphate to bis-phosphoglycerate was addressed. Even at 74 μM FX11, GAPDH activity was not inhibited. Through formal Michaelis-Menten kinetics, the estimated Ki was >>300 μM for GAPDH, indicating that FX11 was selective for LDHA among glycolytic enzymes that use the co-factor NAD.

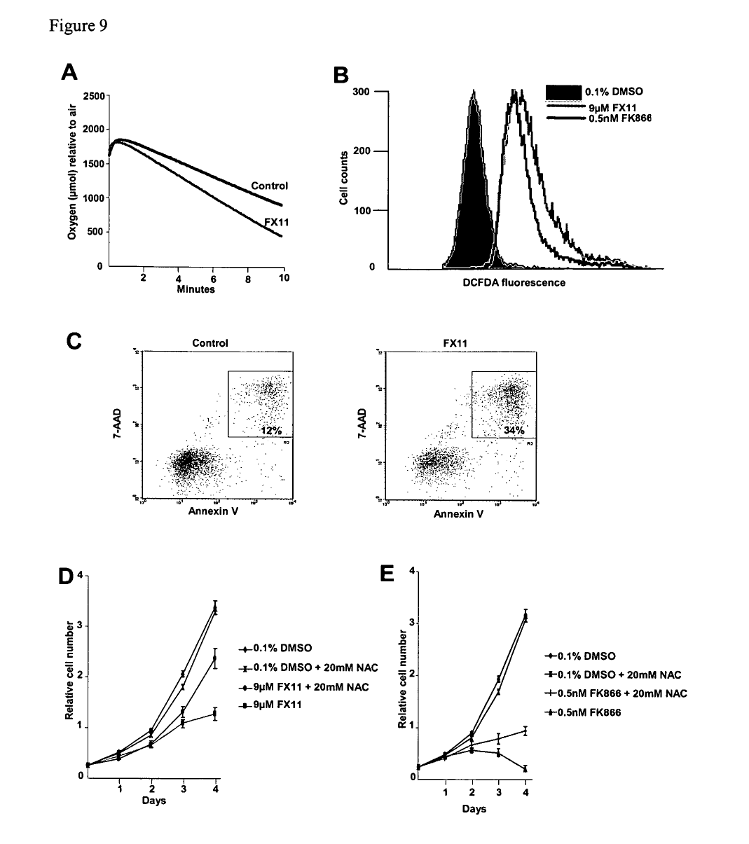

As observed with siRNA-mediated reduction of LDHA, inhibition of LDHA by FX11 also resulted in increased oxygen consumption, ROS production and cell death characterized by increased 7-AAD and annexin V labeling (FIG. 9A-C). The nicotinamide phosphoribosyltransferase (NAMPT) inhibitor of NAD⁺ synthesis, FK 866, also increased ROS production (FIG. 9B). NAC could partially rescue the diminished proliferation of P493 cells treated with either FX11 or FK866 (FIGS. 9D and 9E), indicating that oxidative stress contributed to the inhibition of cell proliferation. Given the significant effect of FX11 on the proliferation of P493 cells, which are dependent on Myc, the trivial possibility that FX11 could inhibit Myc expression itself was ruled out (FIG. 7C). In aggregate, these studies document that reduction of LDHA levels or activity triggers oxidative stress and cell death.

### Example 2

### FX11 Inhibits Glycolysis and Alters Cellular Energy Metabolism

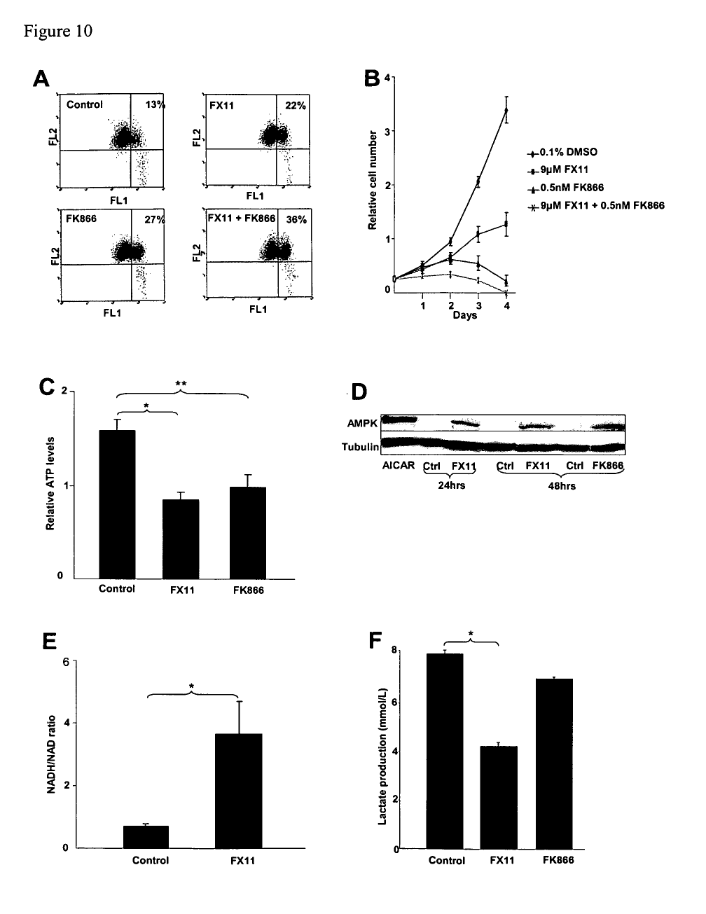

In addition to oxidative stress induced by inhibition of LDHA, it was determined how FX11 affects cellular energy metabolism. First, both FX11 and FK866 decreased mitochondrial membrane potential, and the combination accentuated the abnormality (FIG. 10A). In this regard, the combination of FX11 and FK866 was more toxic to P493 cells than either one alone, causing a more profound inhibition of cell proliferation (FIG. 10B). After 20 hours of exposure to FX11 or FK866, a decrease in ATP levels was accompanied by activation of AMP kinase (AMPK) (FIGS. 10C and 10D), suggesting that in addition to induction of oxidative stress, these agents also deplete cellular energy levels. Decreased ATP levels would further disable cell proliferation, particularly since many cancer cells depend on high levels of ATP production through aerobic glycolysis, in which LDHA recycles NADH back to NAD⁺. Because inhibition of LDHA decreased NAD⁺ recycling, treatment of P493 cells with FX11 was associated with an increase in NADH/NAD⁺ ratio (FIG. 10E). In contrast to FK866, FX11 significantly diminished cellular production of lactate, further supporting LDHA as a biological target of FX11 (FIG. 10F). Treatment with FX11 reduced the conversion of ¹³C-glucose to ¹³C-lactate in P493 cells associated with an increase in ¹³C-glutamate, suggesting that pyruvate was shunted to the mitochondrion and catabolized through the TCA cycle to α-ketoglutarate and in turn to glutamate. Based on these observations, and without wishing to be bound by theory, it is likely that FX11 inhibits glycolysis and shunts pyruvate into the mitochondrion.

### Example 3

### FX11 Inhibits Cells that are Dependent on Glycolysis

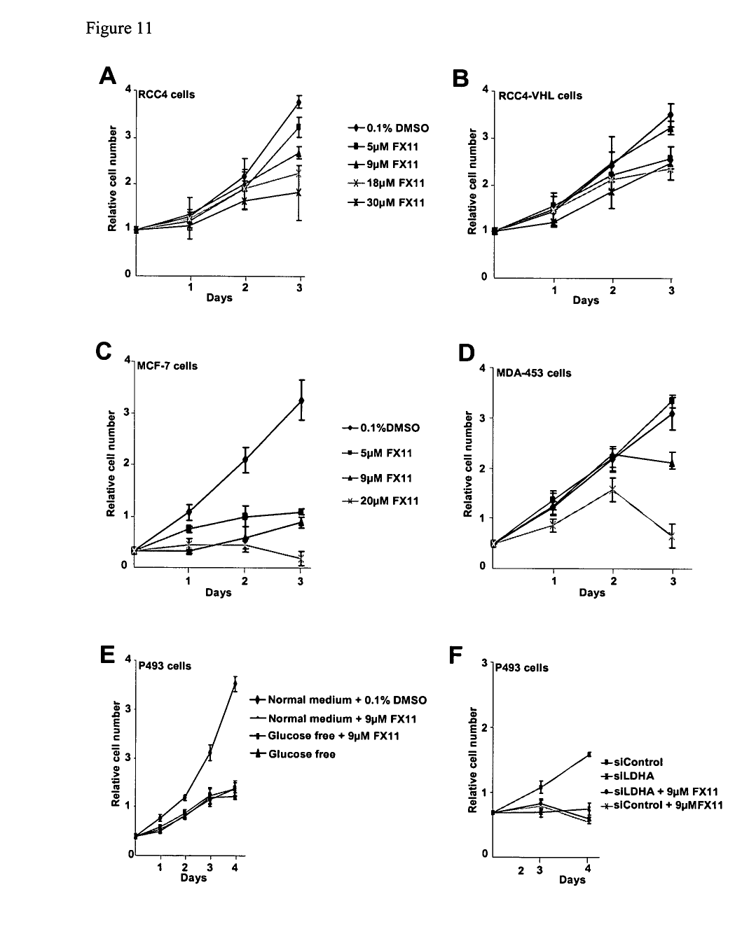

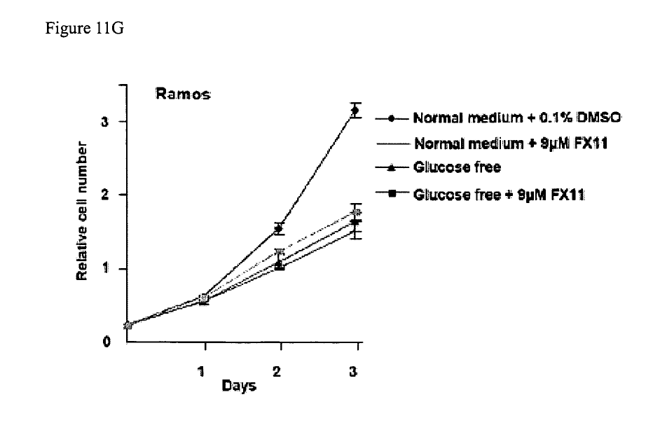

It stands to reason that if FX11 targets LDHA, then cells that depend on LDHA for glycolysis would be more susceptible to FX11 inhibition than those that primarily utilize oxidative phosphorylation. In this regard, to determine whether metabolic phenotypes could affect sensitivity of cancer cells to FX11, the human RCC4 renal cell carcinoma cell line and the RCC4 cell line reconstituted with VHL (RCC4-VHL) were used. Loss of VHL in RCC4 renders these cells constitutively glycolytic due to the stabilization and expression of HIF-1 and HIF-2. Reconstitution with VHL resulted in degradation of HIF-1α and HIF-2α and increased mitochondrial biogenesis and oxygen consumption (Zhang et al., 2007). Given the metabolic differences in these isogenic cell lines, a more significant influence of FX11 on the RCC4 cells was expected as compared with the RCC4-VHL cells. Indeed, a dose-response study revealed that RCC4 was more sensitive to FX11 as compared with RCC4-VHL (FIGS. 11A and 11B).

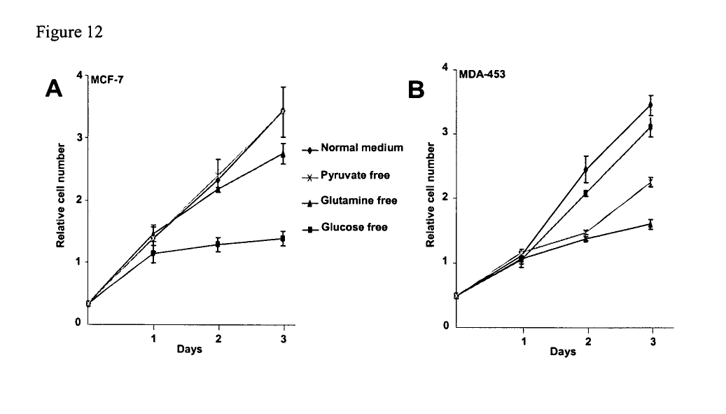

To further corroborate these findings, the glycolytic MCF-7 and the oxidative MDA-MB-453 breast carcinoma cell lines were studied (Mazurek et al., 1997). These studies indicated that MCF-7 was more dependent on glucose, whereas MDA-MB-453 was more dependent on glutamine oxidation (FIGS. 12A and 12B), such that deprivation of glucose has a more profound growth inhibitory effect on MCF-7. A dose-dependent study further revealed that MCF-7 was more sensitive to FX11 (FIGS. 11C and 11D). Although there are many other differences between these cell lines, the correlation of FX11-sensitivity and glucose-dependency of MCF-7 supports the notion that glycolysis predisposes cancer cells to growth inhibition by FX11.

The question of whether the inhibition of human P493 B cells by FX11 depends on glucose or LDHA was addressed. The growth of P493 was inhibited by about 60% when depleted of glucose as compared with growth in 2 mg/L glucose (FIG. 11E). Addition of FX11 could not inhibit P493 cells further in the absence of glucose, suggesting that the effect of FX11 on cell proliferation was glucose-dependent. Furthermore, knock-down of LDHA expression by two sequential electroporations with siRNA caused a markedly diminished proliferative rate that was not further slowed by FX11 (FIG. 11F). FIG. 11G shows that Ramos Burkitt lymphoma cells are also sensitive to FX11. These observations collectively indicated that the growth inhibitory effect of FX11 is consistent with its ability to inhibit LDHA.

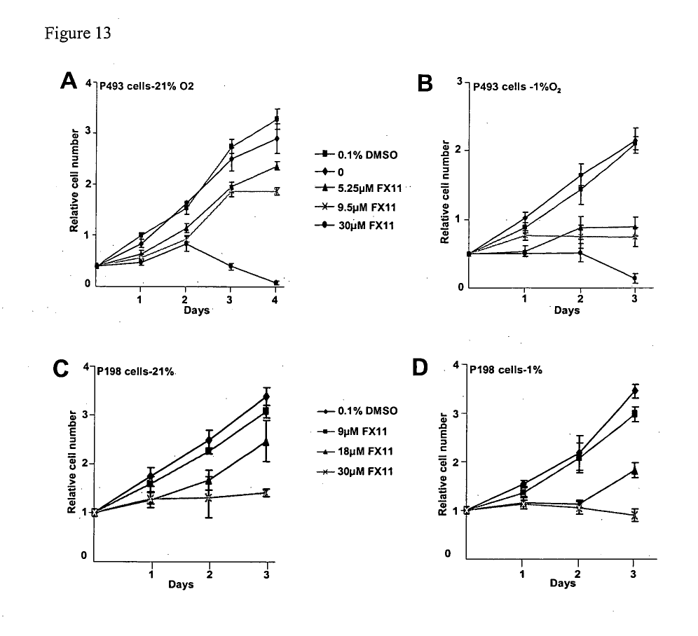

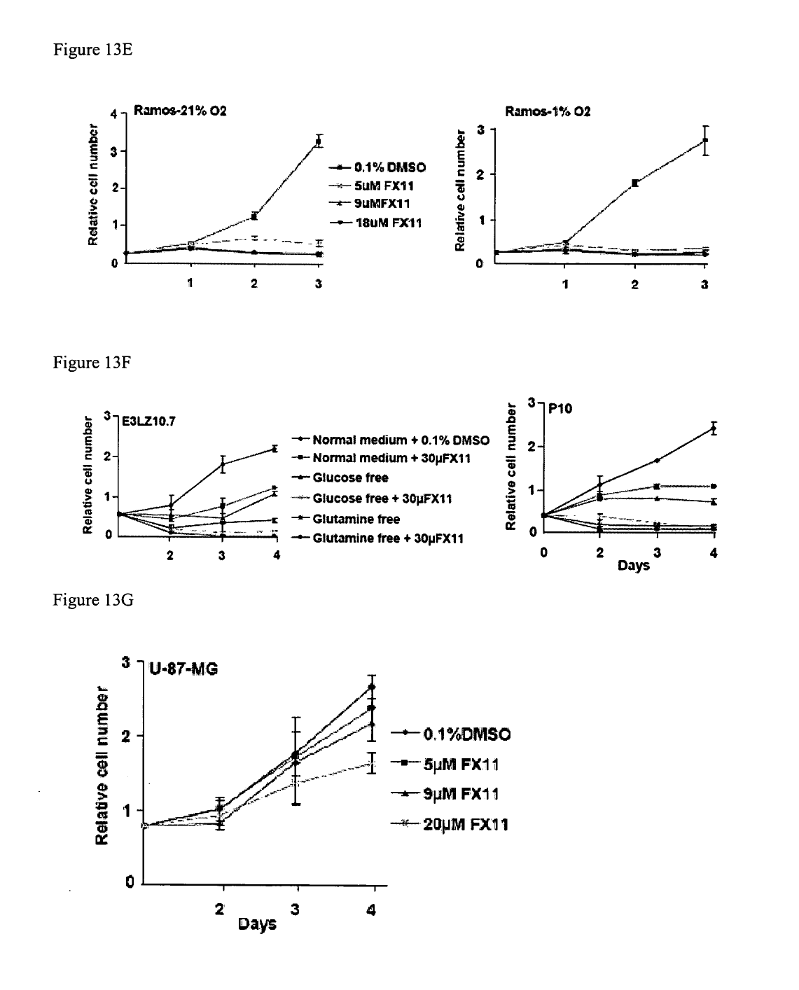

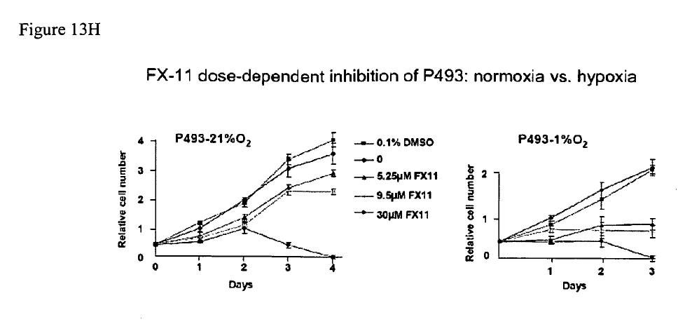

These results suggested that the human P493 B lymphoma cells would be sensitive to FX11 since they express LDHA, but that the sensitivity would be heightened under hypoxia when glycolysis is favored. This is particularly important because P493 cells depend on both glucose and glutamine metabolism when cultured at 20% O₂. When P493 cells were subjected to a dose-response study with FX11, growth inhibition by 9 μM FX11 was increased when the cells were cultured at 1% O₂ (FIGS. 13A and 13B). FX11 inhibited P493 B lymphoma cells in a dose-dependent manner that was augmented in hypoxia. Hypoxia also sensitized the human P198 pancreatic cancer cell line to inhibition by FX11 (FIGS. 13C and 13D), suggesting that reliance on LDHA for hypoxic metabolism causes cancer cells to be susceptible to the growth inhibitory effects of LDHA inhibition by FX11. FIGS. 13E-13H show that FX11 inhibited human Ramos Burkitt lymphoma cell lines, human pancreatic cell lines E3LZ10.7 and P10, human glioblastoma U-87-MG cells, and P493 cells in a dose-dependent manner.

### Example 4

### FX11 Inhibits Tumorigenesis In Vivo

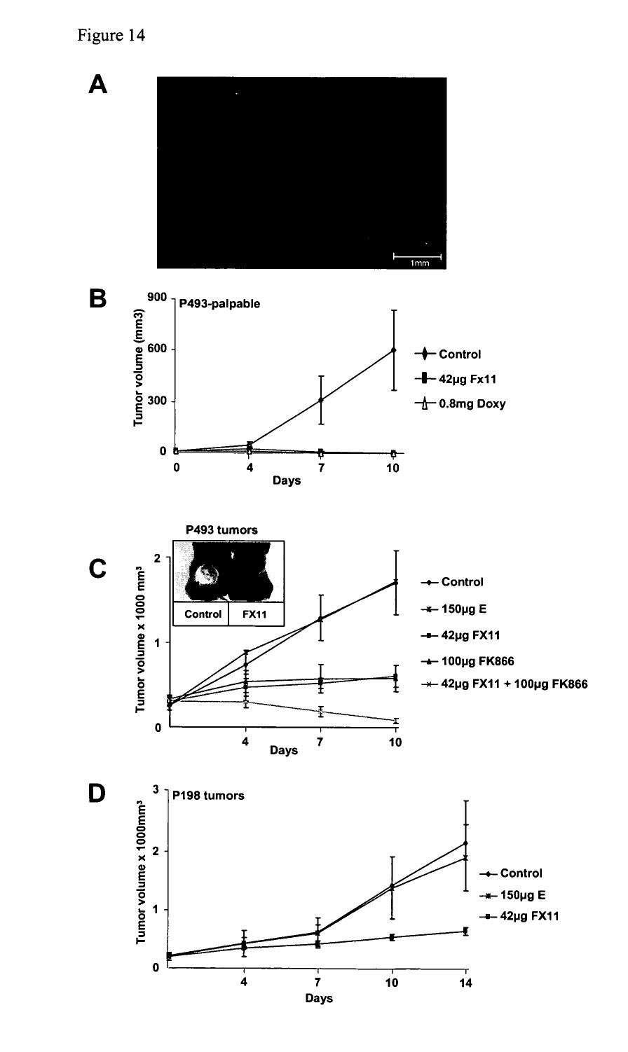

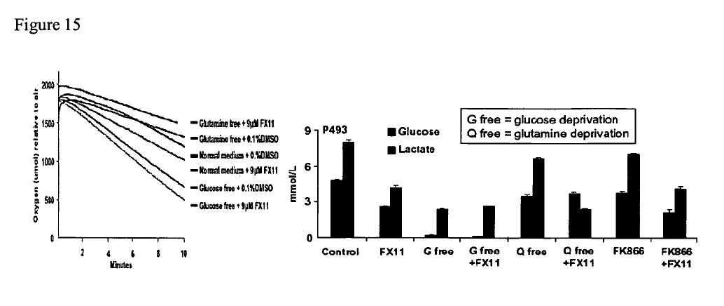

Although the renewed interest in the Warburg effect is accompanied by a greater understanding of its molecular underpinnings, targeting it for therapeutic purposes has become a major challenge. By characterizing a small molecule inhibitor of LDHA, it was found to be effective in inhibiting cellular growth and triggering cell death by both inducing ROS production and depleting ATP. Hypoxia further sensitized human P493 lymphoma cells to LDHA inhibition by FX11. In this regard, the pervasive hypoxic conditions in vivo (FIG. 14A) ought to further force a dependency of these lymphoma cells on glycolysis and particularly on LDHA. Of note, primary human lymphomas have been documented to have elevated LDHA expression particularly in hypoxic regions. Hence, the question of whether in vivo efficacy could be demonstrated with FX11 as an inhibitor of the Warburg effect was examined. A desired FX11 dose of 42 μg for daily intraperitoneal (IP) injection was calculated. An initial serum level of ˜100 μM was expected, assuming a uniform and immediate distribution in the vascular system without accounting for the drug half-life or drug metabolism. It should be noted that solubility has played a significant dose-limiting factor. The dose could only be doubled before the limit of FX11 in aqueous solution was reached.

First, the question of whether FX11 could inhibit P493 lymphomagenesis after a palpable tumor developed was addressed. As controls, animals were injected with vehicle (2% DMSO) or with 0.8 mg doxycycline to inhibit MYC expression in these transformed human B cells. As expected, doxycycline profoundly inhibited tumorigenesis as compared with vehicle injected control animals (FIG. 14B). Intriguingly, daily IP injection of 42 μg of FX11 also resulted in a remarkable inhibition of tumor growth. It is notable that the trivial possibility that FX11 could directly inhibited Myc expression to mediate this profound effect was ruled out (FIG. 14B).

The ability of FX11 to inhibit tumor xenograft growth was challenged by treating P493 lymphomas or human P198 pancreatic tumors with FX11 after the tumor had reached the size of 200 mm³ before treatment commenced. As comparisons, animals were treated with vehicle control or a compound related to FX11, termed E, that lacks the benzyl group and has a Ki for LDHA of >90 μM or more than 10 fold higher than that of FX11. E had no detectable activity as compared with vehicle. FX11 at this solubility-limiting dose displayed a cytostatic, but significant effect over ten days (FIG. 14C). A significant response of human P198 tumor xenografts to FX11 as compared with E was also observed (FIG. 14D). The structure and activity relationship of FX11 and E in vivo correlated with the inhibition and binding of LDHA by these compounds in vitro, further supporting the notion that FX11 targets LDHA.

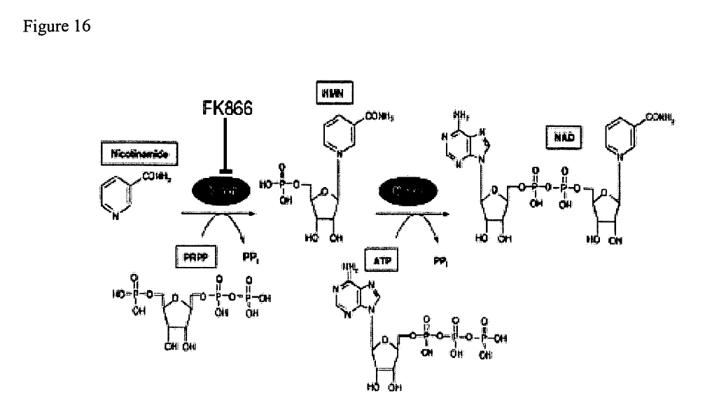

In view of the results reported herein with cultured cells (FIG. 14B), it was predicted that FX11 would synergize with FK866 in the treatment of the P493 human lymphomas. A schematic diagram illustrating FK866 activity is provided in FIG. 16. Hence, a dose of 100 μs of FK866 was selected that gave a cytostatic outcome, and indeed remarkable tumor regression was observed when animals were treated with both FX11 and FK866 (FIG. 14C). These findings underscore the fact that targeting cancer metabolism is feasible and that LDHA is a significant candidate target for further development of FX11 or related compounds.

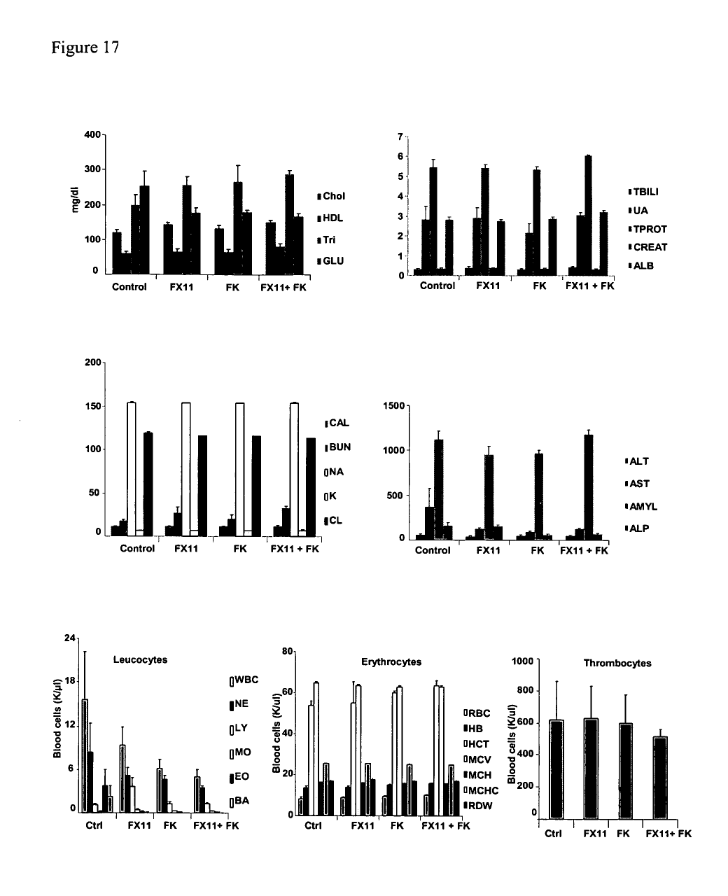

Given the significant effects of FX11 as an LDHA inhibitor in vivo, a group of treated animals was used to address side effects of FX11. As noted above, humans lacking LDHA have been shown to display exertional myopathy. In this regard, although the animals were not formally exercised to examine exertional tolerance, no lethargy or the inability to eat and drink was noted in the animals. In fact, animals treated with FX11 did not lose weight. In initial studies of the hematology and blood chemistry, no cytopenia in animals treated with FX11 alone was observed; however, two (of five studied) animals treated with FK866 did show mild thrombocytopenia. The average leukocyte count in the control group was skewed upward by two animals that had leukocytosis with >15K/μl (normal range 1.8K to 10.7K/μl). The blood chemistries did not reveal any evidence of kidney (BUN, creatinine) or liver (aspartate aminotransferase, alanine aminotransferase, and alkaline phosphatase) toxicity in animals treated with FX11 or FK866 alone or in combination at doses that affected tumor growth in vivo (FIG. 17).

In sum, this work indicates that LDHA inhibition not only resulted in decreased ATP levels and reduced mitochondrial membrane potential, but also a remarkable increase in oxidative stress linked to cell death. This approach indicates that the Warburg effect can be targeted through the use of a small molecule inhibitor of LDHA, termed FX11. Specifically, similar to siRNA reduction of LDHA expression, FX11 could increase cellular oxygen consumption, increase ROS production and induce cell death that could be partially rescued by the anti-oxidant NAC. These observations are corroborated by studies in S. cerevisiae with enhanced or defective respiration (Ruckenstuhl et al., 2009 PLoS ONE 4, e4592). Genetic knock-down or glucose-repression of respiration in yeast reduced apoptosis and enhanced clonogenic survival, whereas forced enhancement of respiration increased ROS production and reduced colony growth that could be partially rescued by the anti-oxidant glutathione. In this regard, a recent perspective on cancer energy metabolism emphasizes the importance of redox homeostasis in cancer cell survival (Vander Heiden et al., 2009 Science 324, 1029-1033). FX11 also increased NADH/NAD+ ratio that was associated with diminished mitochondrial membrane potential and reduced ATP levels. FX11, in contrast to FK866, reduced cellular lactate production. Without wishing to be bound by theory, these results collectively support the notion that FX11 induces cell death through its ability to inhibit LDHA.

Because FX11 has a catechol moiety, it could hypothetically be converted in vivo to a dihydroquinone that is reactive and could cause effects other than inhibition of LDHA. Although the reactive dihydroquinone could also be produced from compound E, it had no detectable anti-tumor activity in vivo. Hence, it is unlikely that conversion of FX11 to a dihydroquinone could account for its anti-tumor activity. As reported herein, tumor xenograft growth in both human B lymphoma and pancreatic cancer xenograft models was effectively inhibited by FX11. At a very large tumor size (200 mm3 in SCID mice that is equivalent to about a kilogram tumor in an adult human), FK866, which is an inhibitor of NAD+ synthesis, was found to synergize with FX11, presumably by augmenting oxidative stress and decreasing ATP production to induce tumor regression in a human lymphoma xenograft model. These studies also document that oxidative stress is an important factor in triggering cell death via inhibiting LDHA, such that anti-oxidant mechanisms (for example, elevated catalase, superoxide dismutase or peroxiredoxins) in cancers will likely play an important role in tumor responses to therapies that target cancer energy metabolism. These in vitro studies demonstrate that differential sensitivities of cells to FX11 depend on their metabolic profile (glycolytic versus oxidative). It stands to reason then, that proper metabolic profiling (for example, dependency on specific energy substrates such as glucose, glutamine or fatty acids or the ability to handle ROS) of cancers will be an essential component for the successful development and implementation of an entirely new class of anti-cancer drugs that targets oncogenic alterations of tumor metabolism.

The results reported herein were obtained using the following methods and materials.

### Cell Lines and Hypoxic Exposure

P493 human lymphoma B cells were maintained in RPMI 1640 with 10% fetal bovine serum (FBS) and 1% penicillin-streptomycin. P198 human pancreatic cancer cells; RCC4, RCC4-VHL human renal carcinoma cells; MCF-7 and MDA-MB-453 breast cancer cells were maintained in high glucose (4.5 mg/ml) DMEM with 10% FBS and 1% penicillin-streptomycin. Non-hypoxic cells (20% O₂) were maintained at 37° C. in a 5% CO₂, 95% in an air incubator. Hypoxic cells (1% O₂) were maintained in a controlled atmosphere chamber (PLAS-LABS, Lansing, Mich.) with a gas mixture containing 1% O₂, 5% CO₂, and 94% N₂ at 37° C. for the indicated time. Bright live cells were counted daily in a hemacytometer using trypan blue dye to exclude dead cells. All cells were grown at a concentration of 10⁵ cells/ml. All drugs treatment began at day 0.

### RNA Interference Experiments

siRNAs targeting human LDHA (ON-TARGETplus SMARTpool) were purchased from Dharmacon Research Inc. Targeting sequences for LDHA were a pool of the following four target sequences: sequence 1, GGAGAAAGCCGUCUUAAUU; sequence 2, GGCAAAGACUAUAAUGUAA; sequence 3, UAAGGGUCUUUACGGAAUA; sequence 4, AAAGUCUUCUGAUGUCAUA. Accordingly, one of skill in the art would recognize the siRNA sequences corresponding to these targets. For P493 human lymphoma B cells, transfection of siRNAs was performed using an Amaxa Nucleotransfection device according to the manufacturer's instructions. Briefly, 2 μg siLDHA ON-TARGETplus SMARTpool or ON-TARGETplus Non-targeting Pool (Dharmacon Research Inc.) were transfected into 2×10⁶ cells at 0 hours. At 24 hours, 10⁵ cells were treated with 0.1% DMSO or FX11 for 48 more hours. The remaining cells were harvested for immunoblot analysis. For P198 human pancreatic cancer cells, transfection of siRNAs was performed using X-tremeGENE siRNA Transfection Reagent (Roche) according to the manufacturer's instructions.

### Western Blot Analysis

Cell pellets were harvested after washing with phosphate buffer saline (PBS). Protein concentration was determined by BCA assay (Pierce) and 30 μg protein per well was separated by SDS-PAGE and transferred by iBlot gel transfer stacks (Invitrogen). Rabbit monoclonal anti-LDHA (Epitomics) was used to detect human LDHA. Membranes were re-probed with anti-α-tubulin as a loading control. The enhanced chemiluminescence reagent ECL (GE Healthcare) was used for detection.

### Oxygen Consumption

Oxygen consumption was measured using a Clark-type oxygen electrode (Oxytherm System, Hansatech Instruments Ltd). 5×10⁶ cells in 1 ml medium were placed in the chamber above a membrane which is permeable to oxygen. Oxygen diffuses through the membrane and is reduced at the cathode surface so that a current flows through the circuit which is completed by a thin layer of KCl solution. The current which is generated bears a direct, stoichiometric relationship to the oxygen reduced, and is converted to a digital signal. Determinations were done in triplicate, and the entire experiment was done twice.

### Reactive Oxygen Species (ROS) Measurement by Flow Cytometry

The measurement of intracellular ROS production was measured by staining cells with 5-(and-6)-carboxy-2′,7′-dichlorodihydrofluorescein diacetate (carboxy-H₂DCFDA; Molecular Probes) according to the manufacturer's instructions. 10⁵ cells/ml were treated with 9 μM FX11 or FK866 for 24 hours. Stained cells were analyzed in FACScan flow cytometers (BD Bioscience).

### Annexin V Assay

After 24 hours of FX11 treatment, cells were harvested and washed twice with cold PBS and the assay was performed using the Annexin V-7-AAD apoptosis detection Kit I (BD Biosciences Pharmingen, San Jose, Calif.) according to the manufacturer's instructions.

### Determination of Ki and Enzyme Kinetics

The reaction velocity of purified human LDHA or GAPDH was determined by a decrease or increase in absorbance at 340 nm of NADH, respectively. The LDHA activity was assessed using the protocol described in Worthington Enzyme Manual: http://www.worthington-biochem.com/LDH/assay.html with varying concentrations of NADH. The GADPH activity was assessed using the protocol described in Worthington Enzyme Manual: http://www.worthington-biochem.com/GAPD/assay.html with varying concentrations of NAD⁺. Ki values were determined from double-reciprocal plots by linear regression analysis using SigmaPlot Enzyme Kinetic software.

### Carboxylink Immobilization and Affinity Columns

FX11 and E molecules which contain carboxyl groups were coupled to immobilized diaminodipropylamine (DADPA) resins according to the manufacturer's instructions (Pierce). About 1.9 mg (95% coupling efficiency) FX11 or E was coupled to 2 ml resin as estimated by amount of either molecule recovered after conjugation. An equal amount of cell lysate was loaded onto these beads and eluted with 1 mM NADH after washing with 6 column volumes of high salt (1 M NaCl). LDHA activity was performed from the eluates to assess the binding affinity of the molecules with LDHA.

### Mitochondrial Membrane Potential Measurement by Flow Cytometry

The lipophilic cation dye (5,5′,6,6′-tetrachloro-1,1′,3,3′-tetraethylbenzimidazolcarbocyanine iodide, JC-1) (Invitrogen) was used to detect the loss of the mitochondrial membrane potential. The negative charge established by the intact mitochondrial membrane potential lets the lipophilic dye stain the mitochondria bright red which emits in channel 2 (FL2). When the mitochondrial membrane potential collapses, JC-1 remains in the cytoplasm in a green fluorescent monomeric emission in channel 1 (FL1). JC-1 reversibly changes its color from green to orange as membrane potentials increase (over values of 80-100 mV). 10⁵ cells/ml were treated with 9 μM FX11 or/and FK866 for 24 hours. Stained cells were analyzed in FACScan flow cytometers (BD Bioscience).

### Measurement of ATP

P493 cells were treated with 9 μM FX11 or 0.5 nM FK866 for 20 hours and counted. ATP levels were determined by luciferin-luciferase-based assay (Promega) on aliquots containing equal number of cells according to standard protocol.

### Determination of NADH/NAD⁺ Ratio

The NADH/NAD⁺ ratios were assayed using EnzyChrom™ NADH/NAD⁺ colorimetric Assay Kit, from BioAssay Systems. This assay is based on an enzyme-catalyzed kinetic reaction where a tetrazolium dye 3-[4,5-dimethylthiazol-2-yl]-2,5-diphenyltetrazolium bromide (MTT) is reduced by NADH in the presence of phenazine methosulfate (PMS). The intensity of the reduced product color, measured at 565 nm, is proportionate to the NADH/NAD⁺ concentration in the sample. Briefly, 10⁵ cells/ml were treated with 9 μM FX11 and grown in tissue culture plates for 24 hours. After washing with cold PBS, cells were homogenized in 100 μl of NAD⁺ or NADH extraction buffer. Following heat extract at 60° C. for 5 minutes, the assay was performed according to the manufacturer's instructions.

### Measurement of Lactate Production

Lactate production was measured by the ABL700 Radiometer analyzer according to the manufacturer's instructions. 10⁵ cells were grown at 37° C. in a 5% CO₂, 95% air incubator and treated with 9 μM FX11 or 0.5 nM FK866 for 24 hours.

### Immunofluorescence Staining

Tumors' hypoxic areas were detected by Pimonidazole Hydrochloride (Hypoxyprobe) from Natural Pharmacia International. Briefly, 1.5 mg Hypoxyprobe diluted in 150 μl of 0.9% saline was given via intraperitoneal injection one hour before tumors were rapidly harvested and fixed in 10% neutral formalin buffer. Aqua DePar and Bord Decloaker RTU (Biocare Medical) were used according to the two-step deparaffinization and heat retrieval protocol of the manufacturer. Protein adducts of reductively-activated pimonidazole were detected by rabbit anti-hypoxyprobe antibody. Samples were analyzed under Zeiss fluorescence microscope at 10× magnification.

### Animal Studies

The animal studies were performed according to the protocols approved by the Animal Care and Use Committee at Johns Hopkins University. In order to generate tumorigenesis study in xenograft model, 2.0×10⁷ P493 human lymphoma B cells or 5×10⁶ human pancreatic cancer cells were injected subcutaneously into male SCID mice (NCI) or athymic Hsd: RH-Foxn1nu mice (Harlan), respectively, as previously described (Feldmann et al., 2007; Gao et al., 2009; Gao et al., 2007). When the tumor volume reached 200 mm³, groups of 5 mice were injected with control 2% DMSO or 42 μg FX11 and/or 100 μg FK866. The tumor volumes were measured using a digital caliper after 4, 7 and 10 days of treatment. Tumor volumes were calculated using the following formula: [length (mm)×width (mm)×width (mm)×0.52]. The entire experiment was repeated seven times.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A composition for the treatment of neoplasia, the composition comprising an effective amount of a compound of Formula III or IV, 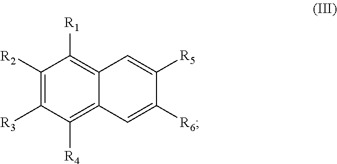
wherein,
R₁ is an optionally substituted alkyl or an optionally substituted aralkyl;
R₂ is H, —C(O)R′, —OR″, or —NR′R″;
R₃ is H, —C(O)R′, —OR″, or —NR′R″;
R₄ is —C(O)R′ or —OR″;
R₅ is an optionally substituted alkyl, an optionally substituted aryl, an optionally substituted heteroaryl, an optionally substituted aralkyl, or an optionally substituted heteroaralkyl;
R₆ is an optionally substituted aralkyl or an optionally substituted heteroaralkyl;
R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
R″ for each occurrence, is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl; and
R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl. 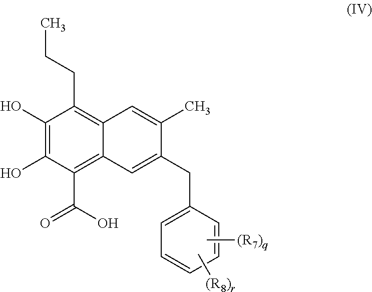
wherein,
each R₇ and R₈ is independently:
(i) an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
(ii) an optionally substituted haloalkyl, cyano, nitro, azido, or halo;
(iii) OR′, SR′, S(O)R′, S(O)₂R′, N(R′)₂, C(O)R′, C(S)R′, C(S)NR′R′, C(NR′)R′, C(NR′)NR′R′, C(O)NR′R′, C(O)NR′OR′, C(O)OR′, OC(O)R′, OC(O)OR′, NR′C(O)NR′R′, NR′C(S)NR′R′, NR′C(O)R′, NR′C(O)OR′, OC(O)NR′R′, or S(O)ᵣNR′R′; or
(iv) R₇ and R₈ may together with the carbon atoms to which each is attached, form a fussed bicyclic aryl, heteroaryl, cycloalkyl, or heterocycloalkyl, each of which may be optionally substituted;
R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
R′″ for each occurrence, is H, an optionally substituted alkyl, an optionally substituted cycloalkyl, an optionally substituted heterocycloalkyl, an optionally substituted aryl, an
optionally substituted heteroaryl, an optionally substituted aralkyl, or an optionally substituted heteroaralkyl;
q is 0, 1, 2, or 3; and
r is 0, 1, or 2.

2. A method for treating neoplasia in a subject, the method comprising administering to said subject an effective amount of a compound that is 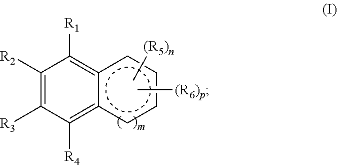
wherein,
R₁ is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, an optionally substituted heteroaralkyl, —C(O)R′, —OR″, —S(O)ₘR′, —NR′R″, or haloalkyl;
R₂ is H, —C(O)R′, —OR″, —S(O)ₘR′, —NR′R″, nitro, cyano, halogen, or haloalkyl;
R₃ is H, —C(O)R′, —OR″, —S(O)ₘR′, —NR′R″, nitro, cyano, halogen, or haloalkyl;
R₄ is H, an optionally substituted alkyl, an optionally substituted aryl, an optionally substituted heteroaryl, an optionally substituted aralkyl, an optionally substituted heteroaralkyl, —C(O)R′, —OR″, —S(O)ₘR′, or —NR′R″;
each R₅ is independently an optionally substituted alkyl, an optionally substituted aryl, an optionally substituted heteroaryl, an optionally substituted aralkyl, or an optionally substituted heteroaralkyl;
each R₆ is independently H, an optionally substituted alkyl, an optionally substituted aryl, an optionally substituted heteroaryl, an optionally substituted aralkyl, or an optionally substituted heteroaralkyl;
R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
R″ for each occurrence, is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
m is 0, 1, or 2;
n is 1 or 2; and
p is 1 or 2. 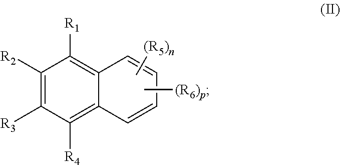
wherein,
R₁ is an optionally substituted alkyl, an optionally substituted aryl, an optionally substituted heteroaryl, an optionally substituted aralkyl, or an optionally substituted heteroaralkyl;
R₂ is H, —C(O)R′, —OR″, —NR′R″, halogen, or haloalkyl;
R₃ is H, —C(O)R′, —OR″, —NR′R″, halogen, or haloalkyl;
R₄ is —C(O)R′, —OR″, —S(O)ₘR′, or —NR′R″;
each R₅ is independently an optionally substituted alkyl, an optionally substituted aryl, an optionally substituted heteroaryl, an optionally substituted aralkyl, or an optionally substituted heteroaralkyl;
each R₆ is independently H, an optionally substituted alkyl, an optionally substituted aryl, an optionally substituted heteroaryl, an optionally substituted aralkyl, or an optionally substituted heteroaralkyl;
R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
R″ for each occurrence, is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
n is 1 or 2; and
p is 1 or 2.

3. A method for treating neoplasia in a subject, the method comprising administering to said subject an effective amount of a compound that is 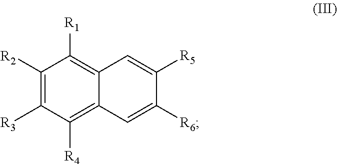
wherein,
R₁ is an optionally substituted alkyl or an optionally substituted aralkyl;
R₂ is H, —C(O)R′, —OR″, or —NR′R″;
R₃ is H, —C(O)R′, —OR″, or —NR′R″;
R₄ is —C(O)R′ or —OR″;
R₅ is an optionally substituted alkyl, an optionally substituted aryl, an optionally substituted heteroaryl, an optionally substituted aralkyl, or an optionally substituted heteroaralkyl;
R₆ is an optionally substituted alkyl, an optionally substituted aryl, an optionally substituted heteroaryl, an optionally substituted aralkyl, or an optionally substituted heteroaralkyl;
R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
R″ for each occurrence, is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl; and
R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl.

4. A method for treating neoplasia in a subject, the method comprising administering to said subject an effective amount of a compound that is 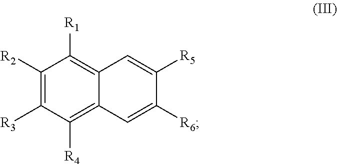
wherein,
R₁ is an optionally substituted alkyl or an optionally substituted aralkyl;
R₂ is H, —C(O)R′, —OR″, or —NR′R″;
R₃ is H, —C(O)R′, —OR″, or —NR′R″;
R₄ is —C(O)R′ or —OR″;
R₅ is an optionally substituted alkyl, an optionally substituted aryl, an optionally substituted heteroaryl, an optionally substituted aralkyl, or an optionally substituted heteroaralkyl;
R₆ is an optionally substituted aralkyl or an optionally substituted heteroaralkyl;
R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
R″ for each occurrence, is H, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl; and
R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl.

5. A method for treating neoplasia in a subject, the method comprising administering to said subject an effective amount of a compound that is 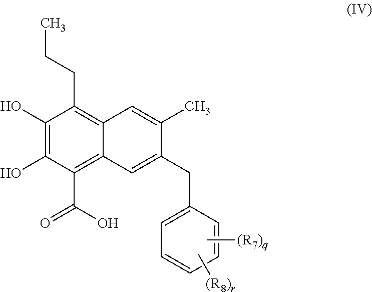
wherein,
each R₇ and R₈ is independently:
(i) an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
(ii) an optionally substituted haloalkyl, cyano, nitro, azido, or halo;
(iii) OR′, SR′, S(O)R′, S(O)₂R′, N(R′)₂, C(O)R′, C(S)R, C(S)NR′R′, C(NR′)R′, C(NR′)NR′R′, C(O)NR′R′, C(O)NR′OR′, C(O)OR′, OC(O)R′, OC(O)OR′, NR′C(O)NR′R′, NR′C(S)NR′R′, NR′C(O)R′, NR′C(O)OR′, OC(O)NR′R′, or S(O)ᵣNR′R′; or
(iv) R₇ and R₈ may together with the carbon atoms to which each is attached, form a fussed bicyclic aryl, heteroaryl, cycloalkyl, or heterocycloalkyl, each of which may be optionally substituted;
R′ for each occurrence, is H, —C(O)R′″, —OR′″, —S(O)ₘR′″, —NR′″R′″, an optionally substituted alkyl, an optionally substituted alkenyl, an optionally substituted alkynyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
R′″ for each occurrence, is H, an optionally substituted alkyl, an optionally substituted cycloalkyl, an optionally substituted heterocycloalkyl, an optionally substituted aryl, an optionally substituted heteroaryl, an optionally substituted aralkyl, or an optionally substituted heteroaralkyl;
q is 0, 1, 2, or 3; and
r is 0, 1, or 2.

6. The method of claim 2, wherein the compound is 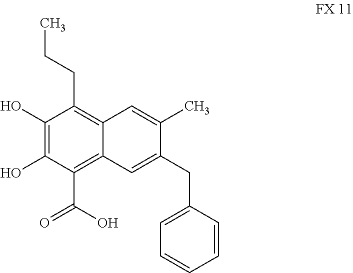
or an analog thereof.

7. A method for treating neoplasia in a subject, the method comprising administering to said subject an effective amount of an agent that selectively inhibits lactate dehydrogenase A activity, thereby treating the neoplasia, or
A method for treating neoplasia in a subject, the method comprising administering to said subject an agent that competitively inhibits the conversion of pyruvate to lactate by lactate dehydrogenase A, thereby treating the neoplasia, or
A method for treating neoplasia in a subject, the method comprising administering to said subject an agent that selectively binds lactate dehydrogenase A, thereby treating the neoplasia.

8-9. (canceled)

10. The method of claim 2, wherein the agent is a compound of Formulas I-IV.

11. The method of claim 2, wherein the compound is FX11, or an analog thereof.

12-15. (canceled)

16. The method of claim 2, wherein the method further comprises administering an effective amount of NAD⁺ synthesis inhibitor FK866.

17. A method for treating a subject having a neoplasm, the method comprising administering to the subject a pharmaceutical composition comprising an effective amount of an agent that reduces the expression or activity of lactate dehydrogenase A and an NAD⁺ synthesis inhibitor.

18. The method of claim 17, wherein the NAD⁺ synthesis inhibitor is FK866.

19. The method of claim 17, wherein the agent is an inhibitory nucleic acid molecule, a compound of Formula I-IV, FX11, or an analog or derivative thereof.

20. The method of claim 19, wherein the inhibitory nucleic acid molecule is an siRNA that targets an LDHA sequence selected from the group consisting of, sequence 1 GGAGAAAGCCGUCUUAAUU; sequence 2, GGCAAAGACUAUAAUGUAA; sequence 3, UAAGGGUCUUUACGGAAUA; sequence 4, AAAGUCUUCUGAUGUCAUA.

21-27. (canceled)

28. A method for selecting a therapeutic regimen for a subject identified as having a neoplasia, the method comprising characterizing a neoplasia as having increased glycolysis relative to a control, wherein said increase indicates that the subject should be treated with a lactate dehydrogenase A inhibitor.

29-31. (canceled)

32. A composition for detecting a neoplasia having increased glycolytic metabolism, the composition comprising a compound of any of Formulas I-IV comprising a detectable moiety.

33. A composition for detecting a neoplasia having increased glycolytic metabolism, the composition comprising FX11 conjugated to a detectable moiety.

34. The composition of claim 32, wherein the detectable moiety is conjugated at R4 of Formula I.

35-37. (canceled)

38. A method for diagnosing a subject as having a neoplasia having increased glycolytic metabolism, the method comprising contacting the subject with an effective amount of a composition of claim 31, and imaging the neoplasia.

39-40. (canceled)

41. A kit for the treatment of a neoplasia, the kit comprising an effective amount of an agent that reduces the expression or activity of lactate dehydrogenase A and directions for the use of the kit for the treatment of a neoplasia, or
A kit for the diagnosis or characterization of a neoplasia, the kit comprising an lactate dehydrogenase A inhibitor comprising a detectable moiety and directions for the use of the kit for the diagnosis or characterization of a neoplasia.

42-45. (canceled)

46. A method for identifying an agent for the treatment of a glycolytic neoplasia, the method comprising
(a) contacting a neoplastic cell that expresses lactate dehydrogenase A with a candidate compound; and
(b) identifying a decrease in lactate dehydrogenase A activity, thereby identifying the agent as useful in the treatment or prevention of a glycolytic neoplasia.

47-50. (canceled)
